SEQUENCE LISTING (41 sequences)

SEQ ID NO: 1 - protein, 119 aa, Archaeoglobus fulgidus
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 2 - protein, 99 aa, Archaeoglobus fulgidus
KVRESGKVKKGTNETTKAVERGLAKLVYIAEDVDPPEIVAHLPLLCEEKNVPYIYVKSKNDLGRAVGIEVPCASAAIINEGELRKELGSLVEKIKGLQK

SEQ ID NO: 3 - protein, 102 aa, Archaeoglobus fulgidus
MYVRFEVPEDMQNEALSLLEKVRESGKVKKGTNETTKAVERGLAKLVYIAEDVDPPEIVAHLPLLCEEKNVPYIYVKSKNDLGRAVGIEVPCASAAIINEGE

SEQ ID NO: 4 - protein, 83 aa, Archaeoglobus fulgidus
MKVRESGKVKKGTNETTKAVERGLAKLVYIAEDVDPPEIVAHLPLLCEEKNVPYIYVKSKNDLGRAVGIEVPCASAAIINEGE

SEQ ID NO: 5 - protein, 151 aa, Pyrobaculum aerophilum
MAVTIDPKTFYANPPPGKPFYVRFEVPNDVAEKALEILSIARQTGKIKKGTNETTKAVERGLAKLVLIAEDVDPPEVVAHLPLLCEEKKVPYVYVPSKEKLGKAAGINVSAAAAVVIEPGQAAGELEALVSKINEVRAKHGLNAIPVPAKR

SEQ ID NO: 6 - protein, 132 aa, Thermoprotei archaeron
MSSSKPFYVRFEVPEELAKKAYEALSKARETGGKIKKGTNETTKAVERGLAKLVLIAEDVDPPEIVAHLPLLCEEKKIPYVYVPSKAQLGKAAGIEVAAASACIIDPGEAKDLVEEIIKEVSEIKVKAGVGK

SEQ ID NO: 7 - protein, 127 aa, Aeropyrum pernix
MSKPIYVRFEVPEDLAEKAYEAVKRARETGRIKKGTNETTKAVERGLAKLVVIAEDVDPPEIVMHLPLLCDEKKIPYVYVPSKKRLGEAAGIEVAAASVAIIEPGDAETLVREIVEKVKELRAKAGV

SEQ ID NO: 8 - protein, 127 aa, Aeropyrum camini
MSKPIYVRFEVPEDLAEKAYEAVKRARETGRIKKGTNETTKAVERGLAKLVVIAEDVDPPEIVMHLPLLCDEKKIPYVYVPSKKRLGEAAGIEVAAASVAIIEPGDADTLVREIIEKVKELRAKAGV

SEQ ID NO: 9 - protein, 155 aa, Pyrobaculum islandicum
MSVTIDPRTFYANPPPGKPFYVRFEVPAEVAEKALEILSIARQTGKIKKGTNETTKAVERGLAKLVLIAEDVDPPEVVAHLPLLCEEKKVPYVYVPSKEKLGKAAGINVSAASAVVIDPGQAAGDLEALVAKINEIRAKHGLNAIPLPSSGAAKK

SEQ ID NO: 10 - protein, 151 aa, Pyrobaculum aerophilum
MAVTIDPKTFYANPPPGKPFYVRFEVPNDVAEKALDILSIARQTGKIKKGTNETTKAVERGLAKLVLIAEDVDPPEVVAHLPLLCEEKKVPYVYVPSKEKLGKAAGINVSAAAAVVIEPGQAAGELEALVSKINEVRAKHGLNAIPVPAKR

SEQ ID NO: 11 - DNA, 360 bp, Archaeoglobus fulgidus
atgtacgtgagatttgaggttcctgaggacatgcagaacgaagctctgagtctgctggagaaggttagggagagcggtaaggtaaagaaaggtaccaacgagacgacaaaggctgtggagaggggactggcaaagctcgtttacatcgcagaggatgttgacccgcctgagatcgttgctcatctgcccctcctctgcgaggagaagaatgtgccgtacatttacgttaaaagcaagaacgaccttggaagggctgtgggcattgaggtgccatgcgcttcggcagcgataatcaacgagggagagctgagaaaggagcttggaagccttgtggagaagattaaaggccttcagaagtaa

SEQ ID NO: 12 - DNA, 300 bp, Archaeoglobus fulgidus
aaggttagggagagcggtaaggtaaagaaaggtaccaacgagacgacaaaggctgtggagaggggactggcaaagctcgtttacatcgcagaggatgttgacccgcctgagatcgttgctcatctgcccctcctctgcgaggagaagaatgtgccgtacatttacgttaaaagcaagaacgaccttggaagggctgtgggcattgaggtgccatgcgcttcggcagcgataatcaacgagggagagctgagaaaggagcttggaagccttgtggagaagattaaaggccttcagaagtaa

SEQ ID NO: 13 - DNA, 300 bp, Archaeoglobus fulgidus
atgtacgtgagatttgaggttcctgaggacatgcagaacgaagctctgagtctgctggagaaggttagggagagcggtaaggtaaagaaaggtaccaacgagacgacaaaggctgtggagaggggactggcaaagctcgtttacatcgcagaggatgttgacccgcctgagatcgttgctcatctgcccctcctctgcgaggagaagaatgtgccgtacatttacgttaaaagcaagaacgaccttggaagggctgtgggcattgaggtgccatgcgcttcggcagcgataatcaacgag

SEQ ID NO: 14 - DNA, 240 bp, Archaeoglobus fulgidus
aaggttagggagagcggtaaggtaaagaaaggtaccaacgagacgacaaaggctgtggagaggggactggcaaagctcgtttacatcgcagaggatgttgacccgcctgagatcgttgctcatctgcccctcctctgcgaggagaagaatgtgccgtacatttacgttaaaagcaagaacgaccttggaagggctgtgggcattgaggtgccatgcgcttcggcagcgataatcaacgag

SEQ ID NO: 15 - DNA, 358 bp, Artificial
  Synthetic
atgtacgtgcggttcgaggtaccggaggacatgcagaatgaggccctgtcacttcttgagaaggtccgagaaagtgggaaagtaaaaaaaggaaccaacgaaacaactaaggcggtcgagagaggacttgccaaattggtttatatagcggaagatgtagatcctccagagatcgttgctcaccttcccttgctttgtgaagagaaaaacgtgccgtatatttacgtgaaaagtaagaacgacctgggccgcgcggttggcatagaggttccttgcgctagtgcggctatcatcaacgagggggaacttaggaaagagctgggatctctcgtggagaagatcaagggacttcaaatag

SEQ ID NO: 16 - protein, 3 aa, Artificial
  Synthetic
SGS

SEQ ID NO: 17 - protein, 3 aa, Artificial
  Synthetic
SGG

SEQ ID NO: 18 - protein, 4 aa, Artificial
  Synthetic
SGGS

SEQ ID NO: 19 - protein, 4 aa, Artificial
  Synthetic
SGGG

SEQ ID NO: 20 - protein, 5 aa, Artificial
  Synthetic
SGGGS

SEQ ID NO: 21 - protein, 5 aa, Artificial
  Synthetic
SGGGG

SEQ ID NO: 22 - protein, 6 aa, Artificial
  Synthetic
SGGGGS

SEQ ID NO: 23 - protein, 6 aa, Artificial
  Synthetic
SGGGGG

SEQ ID NO: 24 - protein, 7 aa, Artificial
  Synthetic
SGGGGGS

SEQ ID NO: 25 - protein, 7 aa, Artificial
  Synthetic
SGGGGGG

SEQ ID NO: 26 - protein, 9 aa, Artificial
  Synthetic
SGGSGGGGS

SEQ ID NO: 27 - protein, 84 aa, Xenopus borealis
QAANYKQLRKGANEATKTLNRGIAEFIVMAADAEPLEIILHLPLLCEDKNVPYVFVRSKQALGRACGVSRPVIACAVTIKEGSQ

SEQ ID NO: 28 - protein, 84 aa, Stylophora pistillata
QASSYKQLRKGANEATKCLNRGLAEFIVMAADTEPLEILLHLPLLCEDKNVPYVFVRSKQALGRACEVSRPVIATAVTINEGSQ

SEQ ID NO: 29 - protein, 84 aa, Drosophila
QALNYNQLRKGANEATKTLNRGLADIVVLAGDTEPIEILMHLPLLCEDKNVPYVFVRSKQALGRACGVSRPIVACSVTTNEGSQ

SEQ ID NO: 30 - protein, 84 aa, Synchytrium microbalum
QASHYKQLKKGANEATKTLNRGIAEFIVMTADTDPLEILLHLPLLCEDKNVPYVFVPSKQALGRACGVSRPVISASVTQNDASE

SEQ ID NO: 31 - protein, 84 aa, Ustilago maydis
QASHYKQLKKGANEATKTLNRGICEFIVMAADVEPIEIVLHLPLLCEDKNVPYVFVPSKTALGRACGVSRPVVSASVTTNEARE

SEQ ID NO: 32 - protein, 84 aa, Ananas comosus
QAANYKQLKKGANEATKTLNRGISEFVVMAADTEPLEILLHLPLLAEDKNVPYVFVPSKQALGRACGVTRPVIACSVTSNEGSQ

SEQ ID NO: 33 - protein, 84 aa, Plasmodium gonderi
RATVYRQLKRGANETVKSLHKGISELVILAADAKPLEIISHIPLVCEDKNTPYVYVRSKMALGRACGISRSVIASSIITKDGSP

SEQ ID NO: 34 - protein, 83 aa, Entamoeba dispar
QAQQNGFVKKGANETTKALNRGKADIVIIAADTKPIEIVLHLPILCEDKNVPYVFVGSKAALGRACGVSRDVIAVAIINDKGK

SEQ ID NO: 35 - protein, 82 aa, Aeropyrum camini
IARDTGKIGKGSNEVTKAVERGDAILVLLAEDVDPPEIIAHMPVLAEEKEIPYIYIPTKDELGEAAGLNVGTASACITDAGE

SEQ ID NO: 36 - protein, 82 aa, Aeropyrum pernix
RARETGRIKKGTNETTKAVERGLAKLVVIAEDVDPPEIVMHLPLLCDEKKIPYVYVPSKKRLGEAAGIEVAAASVAIIEPGD

SEQ ID NO: 37 - protein, 82 aa, Firmicutes; identical to SEQ ID NO: 36

SEQ ID NO: 38 - protein, 82 aa, Thermoprotei archaeon
KARETGKIKKGTNETTKAVERGLAKLVLIAEDVDPPEIVAHLPLLCEEKKIPYVYVPSKAQLGKAAGIEVAAASACIIDPGE

SEQ ID NO: 39 - protein, 81 aa, Pryobaculum aerophilum
IARQTGKIKKGTNETTKAVERGLAKLVLIAEDVDPPEVVAHLPLLCEEKKVPYVYVPSKEKLGKAAGINVSAASAVIDPGQ

SEQ ID NO: 40 - protein, 82 aa, Pryobaculum islandicum
IARQTGKIKKGTNETTKAVERGLAKLVLIAEDVDPPEVVAHLPLLCEEKKVPYVYVPSKEKLGKAAGINVSAASAVVIDPGQ

SEQ ID NO: 41 - protein, 82 aa, Archaeoglobus fulgidus
KVRESGKVKKGTNETTKAVERGLAKLVYIAEDVDPPEIVAHLPLLCEEKNVPYIYVKSKNDLGRAVGIEVPCASAAIINEGE

(19) United States
(12) Patent Application Publication
(10) Pub. No.: US 2024/0024414 A1
(43) Pub. Date: Jan. 25, 2024

(54) ARCHAEA L30 PROTEINS AS UNIVERSAL INFLUENZA VIRUS THERAPEUTICS

(71) Applicant: Icahn School of Medicine at Mount Sinai, New York, NY (US)

(72) Inventor: Benjamin R. Tenoever, New York, NY (US)

(73) Assignee: Icahn School of Medicine at Mount Sinai, New York, NY (US)

(21) Appl. No.: 18/246,014

(22) PCT Filed: Sep. 20, 2021
(86) PCT No.: PCT/US21/51050

§ 371 Date: Mar. 20, 2023

Related U.S. Application Data
(60) Provisional application No. 63/081,032, filed on Sep. 21, 2020.

(51) Int. Cl. A61K 38/16; A61P 31/16; C07K 14/00; C12N 15/86

(52) U.S. Cl. CPC A61K 38/16; A61P 31/16; C07K 14/00; C12N 15/86; A61K 38/00

(57) ABSTRACT

This disclosure provides methods for preventing or treating influenza virus infection or influenza virus diseases in a subject, comprising administering to a subject in need thereof an L7Ae protein or a fragment/variant thereof, or a nucleic acid molecule comprising a polynucleotide encoding an L7Ae protein or a fragment/variant thereof. Also provided are compositions suitable for use in these methods.

### CROSS-REFERENCE TO RELATED APPLICATIONS

This application claims priority under 35 U.S.C. § 119(e) to U.S. Provisional Patent Application No. 63/081,032, filed Sep. 21, 2020. The foregoing application is incorporated by reference herein in its entirety.

### FIELD OF THE INVENTION

This invention relates generally to methods for preventing or treating influenza virus infection or influenza virus diseases using an Archaea L30 protein.

### BACKGROUND OF THE INVENTION

Few things pose a greater threat to humanity than viral pandemics. This threat is greatest amongst viruses with zoonotic potential such as the influenza family of viruses (Krammer, F. et al. Influenza. Nat Rev Dis Primers 4, 3 (2018)). These segmented RNA viruses demonstrate global transmission events annually in birds, humans, and other mammals that lead to natural mutations or complete exchanges of RNA segments that enable immune escape and changes to the transmissibility of the virus. Over the past 100 years, we have witnessed four major influenza virus pandemics, the most serious being the strain that emerged during the turn of the twentieth century that infected one third of the world's population and was responsible for ˜50 million deaths worldwide (Taubenberger, J. K., et al. Sci Transl Med 11 (2019)).

The cornerstone of controlling both pandemic and seasonal influenza virus outbreaks is the development of an effective antiviral and/or a vaccine against the circulating strain. At present, influenza vaccines are developed anew each year in a process that requires six months of lead time demanding we correctly anticipate the dominating strains for the next season (Yamayoshi, S. & Kawaoka, Y. Nat Med 25, 212-220 (2019)). This approach has significant limitations and often results in mismatches between the vaccine and the actual circulating strain(s). For this reason, the National Institutes of Health (NIH) has made one of its highest priorities the development of a universal influenza treatment, including against strains with the potential to cause a pandemic (Paules, C. I., et al. Immunity 47, 599-603 (2017)). The last influenza pandemic occurred in 2009 and was due to an H1N1 Influenza A virus (IAV, H1N1pdm09) that, although relatively mild in nature, demonstrated how unprepared the world was for such an event. Despite enormous efforts to rapidly procure a monovalent H1N1pdm09 vaccine to contain the outbreak, large enough quantities were not available until after the peak of illness. The Centers for Disease Control (CDC) estimated that the outbreak involved 60.8 million cases, 274,304 hospitalizations, and 12,469 deaths (Fineberg, H. V., et al. N Engl J Med 370, 1335-1342 (2014).). While efforts towards a universal vaccine continue to push forward, it still remains unclear whether this will ever be achieved.

In the absence of a universal vaccine, an antiviral that is broadly effective against both seasonal and pandemic strains would make a significant impact in reducing the global burden of influenza virus-based disease. Current therapeutic options for the treatment of influenza virus infection are limited. FDA-approved antiviral drugs for influenza virus include the neuraminidase inhibitors oseltamivir (Tamiflu), zanamivir (Relenza), and peramivir (Rapivab), and the endonuclease inhibitor baloxavir marboxil (Xofluza) (Krammer, F. et al. Nat Rev Dis Primers 4, 3 (2018)). These antivirals show a relatively broad spectrum of activity but also rapidly select for virus-resistant mutants that demonstrate little to no loss of fitness (Hayden, F. G. et al. N Engl J Med 379, 913-923 (2018); Feldmann, F. et al. mBio 10 (2019); Kode, S. S. et al. Virus Res 265, 122-126 (2019)).

Given the global burden imposed by SARS-CoV-2 in the past two years and our proven inability to generate vaccines in a timely manner against H1N1pdm09 in 2009, there is an urgent need to address this vulnerability. Moreover, the lack of a universal vaccine and the capacity of influenza A virus to evade present-day antivirals, the need for a global therapeutic could not be greater.

### SUMMARY OF THE INVENTION

This disclosure addresses the need mentioned above in a number of aspects. In one aspect, this disclosure provides a method for preventing or treating an influenza virus infection or influenza virus disease in a subject. The method comprises administering to a subject in need thereof a nucleic acid molecule comprising a polynucleotide encoding an L7Ae protein (e.g., an Archaeoglobus fulgidus L7Ae protein) or a fragment/variant thereof. In some embodiments, the method comprises administering to the subject in need thereof an L7Ae protein (e.g., an Archaeoglobus fulgidus L7Ae protein) or a fragment/variant thereof.

In some embodiments, the L7Ae protein comprises an amino acid sequence having at least 75% identity to any one of SEQ ID NOs: 1-10, or comprises an amino acid sequence of SEQ ID NOs: 1-10. In some embodiments, the L7Ae protein comprises an amino acid sequence having at least about 75% identify (e.g., at least about any of 80%, 85%, 90%, 95%, 96%, 97%, 98%, 99%, or 100% identity) to SEQ ID NO:4. In some embodiments, the polynucleotide comprises a nucleotide sequence having at least 75% identity to any one of SEQ ID NOs: 11-15, or comprises a nucleotide sequence of SEQ ID NOs: 11-15.

In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:4, wherein the L7Ae protein comprises an I or L at position 88, an E or N at position 89, or a V at position 90, or combinations thereof, wherein the position is relative to SEQ ID NO:1. In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:4, wherein the L7Ae protein comprises an I or L at position 88, an E or N at position 89, and a V at position 90, wherein the position is relative to SEQ ID NO:1. In some embodiments, the L7Ae protein comprises an I at position 88, an E at position 89, and a V at position 90. In some embodiments, the L7Ae protein comprises an I at position 88, an N at position 89, and a V at position 90. In some embodiments, the L7Ae protein comprises an L at position 88, an N at position 89, and a V at position 90. In some embodiments, the L7Ae protein comprises an L at position 88, an E at position 89, and a V at position 90. In some embodiments, the L7Ae protein does not comprise an arginine (R) at position 90. In some embodiments, the L7Ae protein further comprises one or more (such as any of 2, 3, 4, and 5) of amino acid residues N33, E34, K37, R41, and K79, wherein the position is relative to SEQ ID NO:1. In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:4, wherein the L7Ae protein comprises an I or L at position 88, an E or N at position 89, a V at position 90, an N at position 33, an E at position 34, a K at position 37, an R at position 41, and a K at position 79, wherein the position is relative to SEQ ID NO:1.

In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:1, wherein the L7Ae protein comprises an I or L at position 88, an E or N at position 89, or a V at position 90, or combinations thereof, wherein the position is relative to SEQ ID NO:1. In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:1, wherein the L7Ae protein comprises an I or L at position 88, an E or N at position 89, and a V at position 90, wherein the position is relative to SEQ ID NO:1. In some embodiments, the L7Ae protein comprises an I at position 88, an E at position 89, and a V at position 90. In some embodiments, the L7Ae protein comprises an I at position 88, an N at position 89, and a V at position 90. In some embodiments, the L7Ae protein comprises an L at position 88, an N at position 89, and a V at position 90. In some embodiments, the L7Ae protein comprises an L at position 88, an E at position 89, and a V at position 90. In some embodiments, the L7Ae protein does not comprise an arginine (R) at position 90. In some embodiments, the L7Ae protein further comprises one or more (such as any of 2, 3, 4, and 5) of amino acid residues N33, E34, K37, R41, and K79, wherein the position is relative to SEQ ID NO:1. In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:1, wherein the L7Ae protein comprises an I or L at position 88, an E or N at position 89, a V at position 90, an N at position 33, an E at position 34, a K at position 37, an R at position 41, and a K at position 79, wherein the position is relative to SEQ ID NO:1.

In some embodiments, the L7Ae protein comprises an amino acid sequence having at least 63% identity to the amino acid sequence of SEQ ID NO: 4 or SEQ ID NO: 1, or comprises the amino acid sequence of SEQ ID NO: 4 or SEQ ID NO: 1, (a) optionally wherein the L7Ae protein comprises an I or L at position 88, an E or N at position 89, or a V at position 90, or combinations thereof, wherein the position is relative to SEQ ID NO:1, (b) optionally wherein the L7Ae protein comprises an I or L at position 88, an E or N at position 89, and a V at position 90, wherein the position is relative to SEQ ID NO:1, (c) optionally wherein the L7Ae protein comprises an I at position 88, an E at position 89, and a V at position 90, (d) optionally wherein the L7Ae protein comprises an I at position 88, an N at position 89, and a V at position 90, (e) optionally wherein the L7Ae protein comprises an L at position 88, an N at position 89, and a V at position 90, (f) optionally wherein the L7Ae protein comprises an L at position 88, an E at position 89, and a V at position 90, (g) optionally wherein the L7Ae protein does not comprise a R at position 90, (h) optionally wherein the L7Ae protein further comprises one or more of amino acid residues N33, E34, K37, R41, and K79, wherein the position is relative to SEQ ID NO:1, or (i) optionally wherein the L7Ae protein comprises an I or L at position 88, an E or N at position 89, a V at position 90, an N at position 33, an E at position 34, a K at position 37, an R at position 41, and a K at position 79, wherein the position is relative to SEQ ID NO:1.

In some embodiments, the L7Ae protein is at least about any of 80, 85, 90, 95, 100, 105, 110, 115, 120, or 125 amino acids long. In some embodiments, the L7Ae protein is about 80-125 amino acids long, including for example about any of 80-90, 90-100, 100-110, 110-120, and 110-125 amino acids long.

In some embodiments, the nucleic acid molecule described herein comprises a viral vector. In some embodiments, the viral vector comprises an adeno-associated viral vector, lentiviral vector or adenoviral vector. In some embodiments, the adeno-associated viral vector is selected from AAV1, AAV2, AAV3, AAV4, AAV5, AAV6, AAV7, AAV8, AAV9, AAV10, AAV11, AAV 12, AAV13, AAV rh74, and recombinant subtypes thereof.

In some embodiments, the influenza virus infection or influenza virus disease is associated with a virus of the orthomycoviridae family, including, but not limited to, an influenza A virus, an influenza B virus, an influenza C virus, and Isavirus. In some embodiments, the influenza A virus comprises a serotype selected from H1N1, H2N2, H3N1, H3N2, H5N1, H7N7, H1N2, H9N2, H7N2, H7N3, H10N7, H7N9, and H6N1.

In some embodiments, the subject is a mammal. In some embodiments, the subject is human. In some embodiments, the subject is a fish. In some embodiments, the subject is an avian.

In some embodiments, the protein or nucleic acid molecule is administered to the subject prior to the onset of an influenza season. In some embodiments, the method comprises administrating to the subject a second therapeutic agent or therapy. In some embodiments, the second therapeutic agent comprises an antiviral agent. In some embodiments, the antiviral agent is selected from Oseltamivir, Zanamivir, Amantadine, Rimantadine, Arbidol, Laninamivir, Peramivir, Vitamin D, and an interferon. In some embodiments, the second therapeutic agent is administered before, after, or concurrently with administering the nucleic acid molecule.

In another aspect, this disclosure also provides a method for reducing influenza virus replication in a subject or a biological sample thereof. The method comprises: (i) administering to the subject a nucleic acid molecule comprising a polynucleotide encoding an L7Ae protein (e.g., an A. fulgidus L7Ae protein) or a fragment/variant thereof (ii) administering to the subject an L7Ae protein (e.g., an A. fulgidus L7Ae protein) or a fragment/variant thereof or (iii) contacting the biological sample with the nucleic acid molecule or the L7Ae protein or fragment/variant thereof. In some embodiments, the protein or nucleic acid molecule is administered prophylactically or therapeutically.

In some embodiments, the L7Ae protein comprises an amino acid sequence having at least 75% identity to any one of SEQ ID NOs: 1-10, or comprises an amino acid sequence of SEQ ID NOs: 1-10. In some embodiments, the polynucleotide comprises a nucleotide sequence having at least 75% identity to any one of SEQ ID NOs: 11-15, or comprises a nucleotide sequence of SEQ ID NOs: 11-15.

In some embodiments, the nucleic acid molecule comprises a viral vector. In some embodiments, the viral vector comprises an adeno-associated viral vector, lentiviral vector or adenoviral vector. In some embodiments, the adeno-associated viral vector is selected from AAV1, AAV2, AAV3, AAV4, AAV5, AAV6, AAV7, AAV8, AAV9, AAV10, AAV11, AAV 12, AAV13, AAV rh74, and recombinant subtypes thereof.

In some embodiments, the influenza virus infection or influenza virus disease is associated with a virus of the orthomycoviridae family, including, but not limited to, an influenza A virus, an influenza B virus, an influenza C virus, and Isavirus. In some embodiments, the influenza A virus comprises a serotype selected from H1N1, H2N2, H3N1, H3N2, H5N1, H7N7, H1N2, H9N2, H7N2, H7N3, H10N7, H7N9, and H6N1.

In another aspect, this disclosure further provides a method for inhibiting splicing of one or more influenza virus mRNA segments in a subject or a biological sample thereof. The method comprises: (i) administering to the subject a nucleic acid molecule comprising a polynucleotide encoding an L7Ae protein (e.g., an A. fulgidus L7Ae protein) or a fragment/variant thereof (ii) administering to the subject an L7Ae protein, e.g., an A. fulgidus L7Ae protein) or a fragment/variant thereof; or (iii) contacting the biological sample with the protein or the nucleic acid molecule or the L7Ae protein or fragment/variant thereof. In some embodiments, the one or more influenza virus mRNA segments comprise M2 mRNA segment, NS2 mRNA segment, or both.

In yet another aspect, this disclosure additionally provides a vector comprising a polynucleotide encoding an L7Ae protein (e.g., an A. fulgidus L7Ae protein) or a fragment/variant thereof. In some embodiments, the L7Ae protein comprises an amino acid sequence having at least 75% identity to any one of SEQ ID NOs: 1-10, or an amino acid sequence of SEQ ID NOs: 1-10. In some embodiments, the polynucleotide comprises a nucleotide sequence having at least 75% identity to any one of SEQ ID NOs: 11-15, or comprises a nucleotide sequence of SEQ ID NOs: 11-15.

In some embodiments, the vector is a viral vector. In some embodiments, the viral vector is an adeno-associated viral vector, lentiviral vector or adenoviral vector. In some embodiments, the adeno-associated viral vector is selected from AAV1, AAV2, AAV3, AAV4, AAV5, AAV6, AAV7, AAV8, AVV9, AVV10, and recombinant subtypes. In some embodiments, the adeno-associated viral vector is an artificial or recombinant AAV, such recombinant AAV based on any of the AAV subtypes described herein.

Also provided in this disclosure are (a) a host cell comprising the vector described herein; and (b) a composition comprising the vector or the host cell, as described.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

FIGS. 1A, 1B, and 1C (collectively “FIG. 1”) are a set of diagrams showing that Archeae L30 proteins inhibited influenza A viruses (IAV)-mediated splicing. FIG. 1A shows that whole cell lysate from fibroblasts transfected with a library of RNA binding proteins (RBPs) and infected with A/Puerto Rico/8/1934 were analyzed by Western blot for levels of NP, M1, M2, NS1, NS2, the Flag-epitope tagged RBP constructs, and GAPDH. FIG. 1B shows the results of Western blot of whole cell extract from cells transfected with the influenza RNA dependent RNA polymerase, the nucleoprotein (NP), and viral RNA encoding neuraminidase (NA), matrix (M) or the non-structural (NS) proteins. Expression of NA, M1/M2, and NS1/NS2 was assessed by Western blot in the presence of L7Ae. FIG. 1C shows the results of Western blot of whole cell extract derived from fibroblasts expressing GFP or L30 orthologues from the species identified alongside DNA-dependent RNA polymerase-based plasmids expressing NS1 and NS2.

FIGS. 2A, 2B, and 2C (collectively “FIG. 2”) are a set of diagrams depicting the additional results showing that Archeae L30 proteins inhibited IAV-mediated splicing. FIG. 2A shows the results of Western blot of whole cell extract derived from fibroblasts expressing L30 orthologues from the species identified alongside plasmids expressing M1/M2 and either GFP or the L30 orthologue. FIG. 2B shows the results of Western blot of whole cell extract from fibroblasts expressing NP, M1/M2, and NS1/NS2 independent of the influenza replication machinery with and without L7Ae as assessed by NP, M, and NS antibodies. FIG. 2C shows the results of Western blot of whole cell extract derived from plasmids expressing an intronless M1 and NS1 in the presence and absence of L7Ae. FIG. 2D shows the results of Western blot of whole cell extract from fibroblast transfected with L30 members from diverse Archaea alongside a plasmid expressing M1 and M2. FIG. 2E shows the results of Western blot of whole cell extract from fibroblast transfected with L30 members from diverse Archaea alongside a plasmid NS1 and NS2.

FIGS. 3A, 3B, 3C, and 3D (collectively “FIG. 3”) are a set of diagrams showing characterization of Archaea L30 activities. FIG. 3A shows the results of Western blot of whole cell extract derived from fibroblasts co-expressing C- and/or N-terminal truncations of L30 and a plasmid that generates M1/M2. FIG. 3B shows the same Western blot as in FIG. 3A except that truncated L30 proteins were co-expressed with a plasmid expressing NS1 and NS2. FIG. 3C shows the results of Western blot of whole cell extract derived from fibroblasts transfected with a plasmid expressing M1/M2, a dsRed fluorescent gene (DsRed-Myc), DsRed-Myc containing an intron (iDsRed-Myc), and either Flag-epitope tagged GFP or L7Ae. Blot depicts levels of Myc- and Flag-tagged proteins, M1 and M2, and GAPDH. FIG. 3D shows a volcano plot of differentially expressed genes following L7Ae induction. FIG. 3E shows a histogram depicting read alignments for the eight segments of IAV as determined by RNA immunoprecipitation-based sequencing (RIP-Seq) of L7Ae from Archaea, pineapple (Ananas), and candida (Pseudozyma). FIG. 3F shows quantitative RT-PCR of M1/M2 and NS1/NS2 ratios from uninduced (−dox) MDCK cells or MDCK cells expressing L7Ae (+dox) and infected with A/PR/08/34. All data were generated from biological triplicates with error bars denoting standard deviation across samples. ** denotes a p-value less than 0.001 based on Student's t-test.

FIGS. 4A, 4B, 4C, 4D, 4E, and 4F (collectively “FIG. 4”) are a set of diagrams showing the additional results of characterizing Archaea L30 activities. FIG. 4A shows schematic representations of Flag-epitope tagged L30 N- and C-terminal truncations as well as the K37A and K79A (KK) construct. FIG. 4B shows the results of Western blot of whole cell extract derived from fibroblasts co-transfected with either the NS parental segment (NSp) or an NS segment containing an optimized (NSo) 5′ splice site and either Flag-epitope tagged GFP or L7Ae. FIG. 4C shows the results of Western blot of input and immunoprecipitated material from a Flag-based pull down. Blots depict NP and Flag epitope-tagged L7Ae proteins. FIG. 4D shows a volcano plot depicting differentially expressed genes derived from RNA-Seq data associated with FIG. 4C. FIG. 4E shows the results of fluorescent microscopy of GFP, GFP-L30, or L30 constructs containing a nuclear localization sequence (GFP-NLS-L30) or a nuclear export sequence (GFP-NES-L30). FIG. 4F shows Western blot of fibroblasts co-transfected with the L30 constructs in FIG. 4E with either the M or NS segments. Blots depict M1, M2, NS1, NS2, and GFP levels.

FIGS. 5A, 5B, and 5C (collectively “FIG. 5”) are a set of diagrams showing that L7Ae inhibited IAV-mediated splicing. FIG. 5A shows the results of Western blot of whole cell extract derived from wild type MDCK cells or a stable MDCK cell line expressing a doxycycline-inducible L7Ae-2A-YFP cassette infected with influenza A/Puerto Rico/08/1934 (A/PR/8/34) at the indicated hours post infection (hpi) in the absence or present of doxycycline. Blots denote levels of NP, M1, M2, NS1, NS2, and GAPDH. FIG. 5B shows the results of Western blot with the same conditions as in FIG. 5A except the virus used for infections was a modified form of A/PR/8/34, which does not require splicing to generate M2 and NS2. FIG. 5C shows multi-cycle growth curves of the doxycycline-inducible L7Ae-2A-YFP MDCK cells in the absence or presence of Dox in response to influenza A strains (A/California/04/2009 H1N1, A/Netherlands/09/2009 H1N1, A/Texas/36/9 H1N1, A/Panama/99/2007 H3N2, A/Vietnam/1203/2004 H5N1, B/Florida/02/2006, and Vesicular Stomatitis Virus. All data is given in log 2 of plaque-forming units per ml. Error bars represent the standard deviation between samples.

FIG. 6 shows the development of an inducible L7Ae MDCK cell line. The results of fluorescent microscopy of wild type MDCK cells and cells selected to induce L7Ae in the presence of Doxycyclin (+dox) as a L7Ae-2A-YFP construct are shown. YFP was visualized using a green filter.

FIGS. 7A, 7B, and 7C (collectively “FIG. 7”) are a set of diagrams showing an inability to escape L7Ae targeting generates a universal influenza virus therapeutic. FIG. 7A shows the results of Western blot of whole cell extract derived from cells co-expressing Flag epitope-tagged GFP or L7Ae with the various NS segment mutant constructs that were identified after twenty passages of selection. Blots denote levels of NS1, NS2, Flag, and GAPDH. FIG. 7B is a graph denoting the relative fitness of wild type A/PR/8/34 with a virus escape mutant harboring a single nucleotide change at the 5′ splice site of NS2 (g60a). FIG. 7C shows morbidity in mice as denoted by percent weight loss following administration of a control AAV (AAV ctrl) and one expressing L7Ae (AAVL7Ae) and subsequently challenged with 10LD50 of PR/8/34.

FIG. 8 is a graph showing replication properties of A/PR/8/34 NS g60a virus. Multi-cycle growth curve of the recombinant virus selected after 20 passages in L7Ae. This virus, differing from wild type A/PR/8/34, has only a single nucleotide solution in segment 8 (NS g60s) and was titered at 12, 24, 48, and 72 hours in the absence (−dox) or presence (+dox) of L7Ae.

FIGS. 9A and 9B (collectively “FIG. 9”) are a set of diagrams showing that L7Ae expression does not alter the host proteome but reduces IAV M2 and NS2 expression. Mass spectrometry (GC-TOFMS) was performed on IAV-infected human lung epithelial cells which were engineered to express L7Ae in response to doxycycline (+dox). FIG. 9A shows the results of the mass spectrometry-based analyses of biological triplicate samples (comparing no dox quot; −quot; to quot; +doxquot;). FIG. 9B shows the protein level of PB1, PB2, PA, HA, NP, NA, M1, M2, NS1, and NS2.

FIGS. 10A, 10B, 10C, and 10D (collectively “FIG. 10”) are a set of diagrams showing in vivo efficacy of L7Ae. Recombinant full-length L7Ae or bovine serum albumin (BSA) was administered to BL/6 female mice, 4-6 weeks of age. FIG. 10A shows that cohorts of six animals per condition were monitored for weight loss daily (as a proxy for morbidity), and additional three animals were treated per cohort and sacrificed on day three to determine viral load. FIG. 10B shows viral titer in L7Ae-treated animals. FIG. 10C shows body weight of L7Ae-treated animals post infection. FIG. 10D shows mortality across cohorts after a single dose of L7Ae.

FIGS. 11A and 11B (collectively “FIG. 11”) provide sequence alignment and structural depiction of L7Ae proteins. FIG. 11A provides sequence alignment of the core domains of L7Ae orthologues of Archaeoglobus fulgidus that have been tested against influenza A virus with only the members of the Archean domain showing potent inhibition of viral splicing. The amino acid sequences were aligned using MUSCLE v3.8.31, and the resulting alignment was used to construct a neighbor joining phylogenetic tree using the Simple Phylogeny tool at EMBL-EBI (https://www.ebi.ac.uk/Tools/phylogeny/simple_phylogeny/). FIG. 11B provides a cartoon schematic of the crystal structure of the L7Ae-k-turn RNA complex (PDB:1BW0) using PyMOL molecular graphic system.

### DETAILED DESCRIPTION OF THE INVENTION

This present application provides methods for preventing or treating influenza virus infection or influenza virus diseases using an Archaea L30 protein. This disclosure is based, at least in part, on an unexpected discovery that members (e.g., L7Ae) of the archaea L30 family can universally block influenza virus-mediated splicing regardless of strain. Moreover, it was further demonstrated in this disclosure that this activity had no significant impact on the host. Thus, this disclosure provides a new strategy for a universal therapy for seasonal or pandemic influenza threats of the future.

### A. METHODS FOR PREVENTING OR TREATING AN INFLUENZA VIRUS INFECTION

In one aspect, this disclosure provides a method for preventing or treating an influenza virus disease or infection in a subject. The method comprises administering to a subject in need thereof a nucleic acid molecule comprising a polynucleotide encoding an L7Ae protein (e.g., an Archaeoglobus fulgidus L7Ae protein) or a fragment/variant thereof. In some embodiments, the method comprises administering to the subject in need thereof an L7Ae protein (e.g., an Archaeoglobus fulgidus L7Ae protein) or a fragment/variant thereof.

As used herein, the term “influenza virus disease” refers to the pathological state resulting from the presence of an influenza (e.g., influenza A, B or C virus) virus in a cell or subject or the invasion of a cell or subject by an influenza virus. In some embodiments, the term refers to a respiratory illness caused by an influenza virus.

As used herein, the terms “treat,” “treatment,” and “treating” refer in the context of administration of a therapy(ies) to a subject to treat an influenza virus disease or infection to obtain a beneficial or therapeutic effect of a therapy or a combination of therapies. In some embodiments, such terms refer to one, two, three, four, five or more of the following effects resulting from the administration of a therapy or a combination of therapies: (i) the reduction or amelioration of the severity of an influenza virus infection or a disease or a symptom associated therewith; (ii) the reduction in the duration of an influenza virus infection or a disease or a symptom associated therewith; (iii) the regression of an influenza virus infection or a disease or a symptom associated therewith; (iv) the reduction of the titer of an influenza virus; (v) the reduction in organ failure associated with an influenza virus infection or a disease associated therewith; (vi) the reduction in hospitalization of a subject; (vii) the reduction in hospitalization length; (viii) the increase in the survival of a subject; (ix) the elimination of an influenza virus infection or a disease or symptom associated therewith; (x) the inhibition of the progression of an influenza virus infection or a disease or a symptom associated therewith; (xi) the prevention of the spread of an influenza virus from a cell, tissue, organ or subject to another cell, tissue, organ or subject; (xii) the inhibition or reduction in the entry of an influenza virus into a host cell(s); (xiii) the inhibition or reduction in the replication of an influenza virus genome; (xiv) the inhibition or reduction in the synthesis of influenza virus proteins; (xv) the inhibition or reduction in the release of influenza virus particles from a host cell(s); and/or (xvi) the enhancement or improvement the therapeutic effect of another therapy.

The terms “prevent,” “preventing,” “prevention,” “prophylactic treatment” and the like refer to reducing the probability of developing a disorder or condition (e.g., influenza virus disease or infection) in a subject, who does not have, but is at risk of or susceptible to developing a disorder or condition. The term includes prevention of spread of infection in a subject exposed to the virus or at risk of having influenza virus infection.

As used herein, “administering” refers to the physical introduction of a composition comprising a therapeutic agent (e.g., the nucleic acid molecule as described) to a subject, using any of the various methods and delivery systems known to those skilled in the art. Routes of administration described herein include intravenous, intraperitoneal, intramuscular, subcutaneous, spinal or other parenteral routes of administration, for example, by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, a composition described herein can be administered via a non-parenteral route, such as a topical, epidermal or mucosal route of administration, for example, intranasally, orally, vaginally, rectally, sublingually or topically. In some embodiments, the composition is administered intranasally. In some embodiments, the composition is administered orally. In some embodiments, the composition is administered intramuscularly. Administering can also be performed, for example, once, a plurality of times, and/or over one or more extended periods.

The term “nucleic acid,” “nucleotide,” or “polynucleotide” refers to deoxyribonucleic acids (DNA), ribonucleic acids (RNA) and polymers thereof in either single-, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. In some embodiments, a nucleic acid can comprise a mixture of DNA, RNA and analogs thereof. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

In some embodiments, the disclosed L7Ae variant can be encoded by a codon-optimized sequence. For example, the nucleotide sequence encoding the L7Ae variant may be codon-optimized for expression in a eukaryote or eukaryotic cell. In some embodiments, the codon-optimized L7Ae variant is codon-optimized for operability in a eukaryotic cell or organism, e.g., a yeast cell, or a mammalian cell or organism, including a mouse cell, a rat cell, and a human cell or non-human eukaryote organism.

Generally, codon optimization refers to a process of modifying a nucleic acid sequence to enhance expression in the host cells by substituting at least one codon of the native sequence with codons that are more frequently or most frequently used in the genes of that host cell while maintaining the native amino acid sequence. Various species exhibit a particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.). In some embodiments, one or more codons (e.g., 1, 2, 3, 4, 5, 10, 15, 20, 25, 50, or more, or all codons) in a sequence encoding a DNA/RNA-targeting IL-2 variant corresponds to the most frequently used codon for a particular amino acid. As to codon usage in yeast, reference is made to the online Yeast Genome database available at http://www.yeastgenomeorg/community/codonusage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al., Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, pegylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

A peptide or polypeptide “fragment” as used herein refers to a less than full-length peptide, polypeptide or protein. For example, a peptide or polypeptide fragment can have at least about 3, at least about 4, at least about 5, at least about 10, at least about 20, at least about 30, at least about 40 amino acids in length, or single unit lengths thereof. For example, fragment may be 6, 7, 8, 9, 10, 11, 12, 13, 14, 15, 16, 17, or more amino acids in length. There is no upper limit to the size of a peptide fragment. However, in some embodiments, peptide fragments can be less than about 500 amino acids, less than about 400 amino acids, less than about 300 amino acids or less than about 250 amino acids in length.

Also within the scope of this disclosure are the variants, mutants, and homologs with significant identity to the L7Ae protein. For example, such variants and homologs may have sequences with at least about about 63%, about 64%, about 65%, about 66%, about 67%, about 68%, about 69%, 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% sequence identity with the sequences of L7Ae described herein.

In some embodiments, the L7Ae protein comprises an amino acid sequence having at least 75% identity (e.g., 80%, 85%, 90%, 95%, 96%, 97%, 98%, 99%) to any one of SEQ ID NOs: 1-10, or comprises an amino acid sequence of SEQ ID NOs: 1-10.

In some embodiments, the polynucleotide comprises a nucleotide sequence having at least 75% (e.g., 80%, 85%, 90%, 95%, 96%, 97%, 98%, 99%) identity to any one of SEQ ID NOs: 11-15, or comprises a nucleotide sequence of SEQ ID NOs: 11-15.

TABLE 1
REPRESENTATIVE SEQUENCES

| | | | | % identify |
|---|---|---|---|---|
| SEQ | | | % identity | to SEQ ID |
| ID | | OTHER | of SEQ ID | NO: 4 (aa |
| NO | SEQUENCES | INFORMATION | NO: 1 | 21-102) |
| 1 | MYVRFEVPEDMQNEALSLLEKVR | Archaeoglobus | 100% | 100% |
| | ESGKVKKGTNETTKAVERGLAKL | fulgidus | | |
| | VYIAEDVDPPEIVAHLPLLCEEKN | L7Ae | | |
| | VPYIYVKSKNDLGRAVGIEVPCAS | Full-length (119 | | |
| | AAIINEGELRKELGSLVEKIKGLQ | residues) | | |
| | K | | | |
| 2 | MKVRESGKVKKGTNETTKAVER | Archaeoglobus | 82.3% | 100% |
| | GLAKLVYIAEDVDPPEIVAHLPLL | fulgidus | | |
| | CEEKNVPYIYVKSKNDLGRAVGI | L7Ae | | |
| | EVPCASAAIINEGELRKELGSLVE | ΔN-21 | | |
| | KIKGLQK | | | |
| 3 | MYVRFEVPEDMQNEALSLLEKVR | Archaeoglobus | 77.% | 100% |
| | ESGKVKKGTNETTKAVERGLAKL | fulgidus | | |
| | VYIAEDVDPPEIVAHLPLLCEEKN | L7Ae | | |
| | VPYIYVKSKNDLGRAVGIEVPCAS | ΔC-27 | | |
| | AAIINEGE | | | |
| 4 | MKVRESGKVKKGTNETTKAVER | Archaeoglobus | 68% | 100% |
| | GLAKLVYIAEDVDPPEIVAHLPLL | fulgidus | | |
| | CEEKNVPYIYVKSKNDLGRAVGI | L7Ae | | |
| | EVPCASAAIINEGE | 21-102 | | |
| 5 | MAVTIDPKTFYANPPPGKPFYVRF | Pyrobaculum | 67% | 74% |
| | EVPNDVAEKALEILSIARQTGKIK | aerophilum | | |
| | KGTNETTKAVERGLAKLVLIAED | L7Ae | | |
| | VDPPEVVAHLPLLCEEKKVPYVY | | | |
| | VPSKEKLGKAAGINVSAAAAVVI | | | |
| | EPGQAAGELEALVSKINEVRAKH | | | |
| | GLNAIPVPAKR | | | |
| 6 | MSSSKPFYVRFEVPEELAKKAYE | Thermoprotei | 69% | 78% |
| | ALSKARETGGKIKKGTNETTKAV | archaeron | | |
| | ERGLAKLVLIAEDVDPPEIVAHLP | L7Ae | | |
| | LLCEEKKIPYVYVPSKAQLGKAA | | | |
| | GIEVAAASACIIDPGEAKDLVEEII | | | |
| | KEVSEIKVKAGVGK | | | |
| 7 | MSKPIYVRFEVPEDLAEKAYEAV | Aeropyrum | 64% | 73% |
| | KRARETGRIKKGTNETTKAVERG | pernix | | |
| | LAKLVVIAEDVDPPEIVMHLPLLC | L7Ae | | |
| | DEKKIPYVYVPSKKRLGEAAGIEV | | | |
| | AAASVAIIEPGDAETLVREIVEKV | | | |
| | KELRAKAGV | | | |
| 8 | MSKPIYVRFEVPEDLAEKAYEAV | Aeropyrum | 63% | 73% |
| | KRARETGRIKKGTNETTKAVERG | camini | | |
| | LAKLVVIAEDVDPPEIVMHLPLLC | L7Ae | | |
| | DEKKIPYVYVPSKKRLGEAAGIEV | | | |
| | AAASVAIIEPGDADTLVREIIEKVK | | | |
| | ELRAKAGV | | | |
| 9 | MSVTIDPRTFYANPPPGKPFYVRF | Pyrobaculum | 66% | 75% |
| | EVPAEVAEKALEILSIARQTGKIK | islandicum | | |
| | KGTNETTKAVERGLAKLVLIAED | L7Ae | | |
| | VDPPEVVAHLPLLCEEKKVPYVY | | | |
| | VPSKEKLGKAAGINVSAASAVVI | | | |
| | DPGQAAGDLEALVAKINEIRAKH | | | |
| | GLNAIPLPSSGAAKK | | | |
| 10 | MAVTIDPKTFYANPPPGKPFYVRF | Pyrobaculum | 67% | 74% |
| | EVPNDVAEKALDILSIARQTGKIK | aerophilum | | |
| | KGTNETTKAVERGLAKLVLIAED | L7Ae | | |
| | VDPPEVVAHLPLLCEEKKVPYVY | | | |
| | VPSKEKLGKAAGINVSAAAAVVI | | | |
| | EPGQAAGELEALVSKINEVRAKH | | | |
| | GLNAIPVPAKR | | | |
| 11 | atgtacgtgagatttgaggttcctgaggacatgcaga | Archaeoglobus | N/A | N/A |
| | acgaagctctgagtctgctggagaaggttagggag | fulgidus | | |
| | agcggtaaggtaaagaaaggtaccaacgagacga | L7Ae | | |
| | caaaggctgtggagaggggactggcaaagctcgtt | Full-length | | |
| | tacatcgcagaggatgttgacccgcctgagatcgttg | | | |
| | ctcatctgcccctcctctgcgaggagaagaatgtgcc | | | |
| | gtacatttacgttaaaagcaagaacgaccttggaagg | | | |
| | gctgtgggcattgaggtgccatgcgcttcggcagcg | | | |
| | ataatcaacgagggagagctgagaaaggagcttgg | | | |
| | aagccttgtggagaagattaaaggccttcagaagta | | | |
| | a | | | |
| 12 | atgaaggttagggagagcggtaaggtaaagaaagg | Archaeoglobus | N/A | N/A |
| | taccaacgagacgacaaaggctgtggagagggga | fulgidus | | |
| | ctggcaaagctcgtttacatcgcagaggatgttgacc | L7Ae | | |
| | cgcctgagatcgttgctcatctgcccctcctctgcga | ΔN-21 | | |
| | ggagaagaatgtgccgtacatttacgttaaaagcaa | | | |
| | gaacgaccttggaagggctgtgggcattgaggtgc | | | |
| | catgcgcttcggcagcgataatcaacgagggagag | | | |
| | ctgagaaaggagcttggaagccttgtggagaagatt | | | |
| | aaaggccttcagaagtaa | | | |
| 13 | atgtacgtgagatttgaggttcctgaggacatgcaga | Archaeoglobus | N/A | N/A |
| | acgaagctctgagtctgctggagaaggttagggag | fulgidus | | |
| | agcggtaaggtaaagaaaggtaccaacgagacga | L7Ae | | |
| | caaaggctgtggagaggggactggcaaagctcgtt | ΔC-27 | | |
| | tacatcgcagaggatgttgacccgcctgagatcgttg | | | |
| | ctcatctgcccctcctctgcgaggagaagaatgtgcc | | | |
| | gtacatttacgttaaaagcaagaacgaccttggaagg | | | |
| | gctgtgggcattgaggtgccatgcgcttcggcagcg | | | |
| | ataatcaacgag | | | |
| 14 | atgaaggttagggagagcggtaaggtaaagaaagg | Archaeoglobus | N/A | N/A |
| | taccaacgagacgacaaaggctgtggagagggga | fulgidus | | |
| | ctggcaaagctcgtttacatcgcagaggatgttgacc | L7Ae | | |
| | cgcctgagatcgttgctcatctgcccctcctctgcga | 21-102 | | |
| | ggagaagaatgtgccgtacatttacgttaaaagcaa | | | |
| | gaacgaccttggaagggctgtgggcattgaggtgc | | | |
| | catgcgcttcggcagcgataatcaacgag | | | |
| 15 | atgtacgtgcggttcgaggtaccggaggacatgca | Archaeoglobus | N/A | N/A |
| | gaatgaggccctgtcacttcttgagaaggtccgaga | fulgidus | | |
| | aagtgggaaagtaaaaaaaggaaccaacgaaaca | L7Ae (codon- | | |
| | actaaggcggtcgagagaggacttgccaaattggtt | optimized) | | |
| | tatatagcggaagatgtagatcctccagagatcgttg | | | |
| | ctcaccttcccttgctttgtgaagagaaaaacgtgcc | | | |
| | gtatatttacgtgaaaagtaagaacgacctgggccg | | | |
| | cgcggttggcatagaggttccttgcgctagtgcggct | | | |
| | atcatcaacgagggggaacttaggaaagagctggg | | | |
| | atctctcgtggagaagatcaagggacttcaaatag | | | |

The term “homolog” or “homologous,” when used in reference to a polypeptide, refers to a high degree of sequence identity between two polypeptides, or to a high degree of similarity between the three-dimensional structure or to a high degree of similarity between the active site and the mechanism of action. In some embodiments, a homolog has a greater than 60% sequence identity, and more preferably greater than 75% sequence identity, and still more preferably greater than 90% sequence identity, with a reference sequence. The term “substantial identity,” as applied to polypeptides, means that two peptide sequences, when optimally aligned, such as by the programs GAP or BESTFIT using default gap weights, share at least 75% sequence identity.

As used herein, the term “variant” refers to a first composition (e.g., a first molecule) that is related to a second composition (e.g., a second molecule, also termed a “parent” molecule). The variant molecule can be derived from, isolated from, based on or homologous to the parent molecule. The term variant can be used to describe either polynucleotides or polypeptides.

As applied to polynucleotides, a variant molecule can have an entire nucleotide sequence identity with the original parent molecule, or alternatively, can have less than 100% nucleotide sequence identity with the parent molecule. For example, a variant of a gene nucleotide sequence can be a second nucleotide sequence that is at least 50%, 60%, 70%, 80%, 90%, 95%, 98%, 99% or more identical in nucleotide sequence compare to the original nucleotide sequence. Polynucleotide variants also include polynucleotides comprising the entire parent polynucleotide, and further comprising additional fused nucleotide sequences. Polynucleotide variants also include polynucleotides that are portions or subsequences of the parent polynucleotide, for example, unique subsequences (e.g., as determined by standard sequence comparison and alignment techniques) of the polynucleotides disclosed herein are also encompassed by the invention.

In another aspect, polynucleotide variants include nucleotide sequences that contain minor, trivial or inconsequential changes to the parent nucleotide sequence. For example, minor, trivial or inconsequential changes include changes to nucleotide sequence that (i) do not change the amino acid sequence of the corresponding polypeptide, (ii) occur outside the protein-coding open reading frame of a polynucleotide, (iii) result in deletions or insertions that may impact the corresponding amino acid sequence, but have little or no impact on the biological activity of the polypeptide, (iv) the nucleotide changes result in the substitution of an amino acid with a chemically similar amino acid. In the case where a polynucleotide does not encode for a protein (for example, a tRNA or a crRNA or a tracrRNA), variants of that polynucleotide can include nucleotide changes that do not result in loss of function of the polynucleotide. In another aspect, conservative variants of the disclosed nucleotide sequences that yield functionally identical nucleotide sequences are encompassed by the invention. One of skill will appreciate that many variants of the disclosed nucleotide sequences are encompassed by the invention.

As applied to proteins, a variant polypeptide can have an entire amino acid sequence identity with the original parent polypeptide, or alternatively, can have less than 100% amino acid identity with the parent protein. For example, a variant of an amino acid sequence can be a second amino acid sequence that is at least 50%, 60%, 70%, 80%, 90%, 95%, 98%, 99% or more identical in amino acid sequence compared to the original amino acid sequence.

Polypeptide variants include polypeptides comprising the entire parent polypeptide, and further comprising additional fused amino acid sequences. Polypeptide variants also include polypeptides that are portions or subsequences of the parent polypeptide, for example, unique subsequences (e.g., as determined by standard sequence comparison and alignment techniques) of the polypeptides disclosed herein are also encompassed by the invention. For example, in some embodiments, the L7Ae protein comprises one or more mutations relative to a corresponding wildtype protein. In some embodiments, the L7Ae protein comprises a mutation at the K37 and/or K79 position (e.g., K37A and K79A mutations), wherein the position is relative to SEQ ID NO:1.

In another aspect, polypeptide variants include polypeptides that contain minor, trivial, or inconsequential changes to the parent amino acid sequence. For example, minor, trivial, or inconsequential changes include amino acid changes (including substitutions, deletions, and insertions) that have little or no impact on the biological activity of the polypeptide, and yield functionally identical polypeptides, including additions of non-functional peptide sequence. In other aspects, the variant polypeptides of the invention change the biological activity of the parent molecule. One of skill will appreciate that many variants of the disclosed polypeptides are encompassed by the invention.

In some aspects, polynucleotide or polypeptide variants of the invention can include variant molecules that alter, add or delete a small percentage of the nucleotide or amino acid positions, for example, typically less than about 10%, less than about 5%, less than 4%, less than 2% or less than 1%.

For example, in some embodiments, the L7Ae protein comprises no more than 10 (such as any of one, two, three, four, five, six, seven, eight, nine, or 10) amino acid substitutions relative to a wildtype L7Ae protein, wherein the wildtype L7Ae protein comprises the amino acid sequence of any one of SEQ ID NOs:1-10. In some embodiments, the L7Ae protein comprises no more than 10 (such as any of one, two, three, four, five, six, seven, eight, nine, or 10) amino acid substitutions relative to SEQ ID NO:4.

A “functional variant” of a protein as used herein refers to a variant of such protein that retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide or peptide. Functional variants may be naturally occurring or may be man-made.

In some embodiments, a variant of a L7Ae protein may include one or more conservative modifications. The L7Ae protein variant with one or more conservative modifications may retain the desired functional properties, which can be tested using the functional assays known in the art.

As used herein, the term “conservative sequence modifications” refers to amino acid modifications that do not significantly affect or alter the binding characteristics of the protein containing the amino acid sequence. Such conservative modifications include amino acid substitutions, additions, and deletions. Modifications can be introduced by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include: amino acids with basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine, tryptophan); nonpolar side chains (e.g., alanine, valine, leucine, isoleucine, proline, phenylalanine, methionine); beta-branched side chains (e.g., threonine, valine, isoleucine); and aromatic side chains (e.g., tyrosine, phenylalanine, tryptophan, histidine) includes one or more conservative modifications. The L7Ae protein with one or more conservative modifications may retain the desired functional properties, which can be tested using the functional assays known in the art.

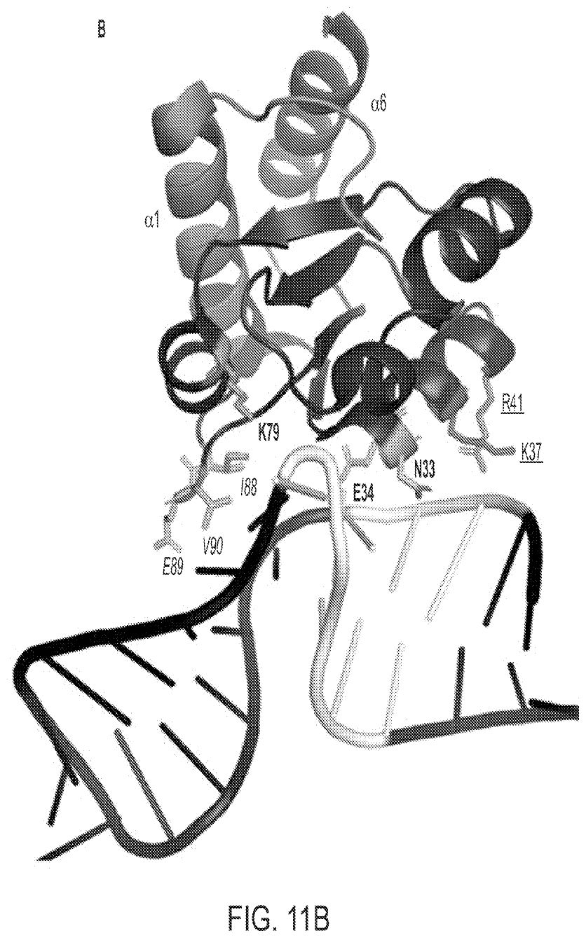

FIG. 11A provides sequence alignment of the core domains of L7Ae orthologues of Archaeoglobus fulgidus that have been tested against influenza A virus with only the members of the Archean domain showing potent inhibition of viral splicing. Sequence identity was determined based on a standard BLASTp search using the default parameters set when two or more sequences aligned together. The values included are those defined as ‘Identities” by NCBI and do not account for residues that are not conserved but share similar properties (Positives’ as defined by NCBI). Unless indicated otherwise, sequence identities described herein refer to sequence identities obtained under such conditions. Sequence identity can be expressed either in terms of a protein or polynucleotide having a certain percentage of sequence identity compared to a reference sequence, or in terms of a protein or polynucleotide that is certain percent identical to a reference sequence.

As shown in FIG. 11A, one key distinction that sets the Archean L7Ae family apart is the conservation at positions 88, 89, and 90. The conserved residues are located in the RNA binding “knuckle” (as depicted in FIG. 11B), and are leucine (L) or isoleucine (I) at position 88, glutamine acid (E) or asparagine (N) at position 89, and a valine (V) as position 90. In addition, the sequence alignment shows that residues N33, E34, K37, R41, and K79 are highly conserved among all L7Ae proteins. These residues are implicated as critical RNA binding contacts according to the protein structure. L7Ae proteins comprising one or a combination of these conserved residues are therefore particularly useful for methods described herein.

In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:1, wherein the L7Ae protein comprises an I or L at position 88, an E or N at position 89, or a V at position 90, or combinations thereof, wherein the position is relative to SEQ ID NO:1. In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:1, wherein the L7Ae protein comprises an I or L at position 88, an E or N at position 89, and a V at position 90, wherein the position is relative to SEQ ID NO:1. In some embodiments, the L7Ae protein comprises an I at position 88, an E at position 89, and a V at position 90. In some embodiments, the L7Ae protein comprises an I at position 88, an N at position 89, and a V at position 90. In some embodiments, the L7Ae protein comprises an L at position 88, an N at position 89, and a V at position 90. In some embodiments, the L7Ae protein does not comprise an arginine (R) at position 90. In some embodiments, the L7Ae protein further comprises one or more (such as any of 2, 3, 4, and 5) of amino acid residues N33, E34, K37, R41, and K79, wherein the position is relative to SEQ ID NO: 1. In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:1, wherein the L7Ae protein comprises an I or L at position 88, an E or N at position 89, a V at position 90, an N at position 33, an E at position 34, a K at position 37, an R at position 41, and a K at position 79, wherein the position is relative to SEQ ID NO:1.

In some embodiments, the L7Ae protein comprises no more than 10 (such as any of zero, one, two, three, four, five, six, seven, eight, nine, or 10) amino acid substitutions relative to a wildtype L7Ae protein, wherein the wildtype L7Ae protein comprises the amino acid sequence of any one of SEQ ID Nos:1-10, wherein the L7Ae protein comprises an I or L at position 88, an E or N at position 89, or a V at position 90, or combinations thereof, wherein the position is relative to SEQ ID NO:1. In some embodiments, the L7Ae protein comprises no more than 10 (such as any of zero, one, two, three, four, five, six, seven, eight, nine, or 10) amino acid substitutions relative to a wildtype L7Ae protein, wherein the wildtype L7Ae protein comprises the amino acid sequence of any one of SEQ ID Nos:1-10, wherein the L7Ae protein comprises an I or L at position 88, an E or N at position 89, and a V at position 90, wherein the position is relative to SEQ ID NO:1. In some embodiments, the L7Ae protein comprises an I at position 88, an E at position 89, and a V at position 90. In some embodiments, the L7Ae protein comprises an I at position 88, an N at position 89, and a V at position 90. In some embodiments, the L7Ae protein comprises an L at position 88, an N at position 89, and a V at position 90. In some embodiments, the L7Ae protein does not comprise an arginine (R) at position 90. In some embodiments, the L7Ae protein further comprises one or more (such as any of 2, 3, 4, and 5) of amino acid residues N33, E34, K37, R41, and K79, wherein the position is relative to SEQ ID NO: 1. In some embodiments, the L7Ae protein comprises no more than 10 (such as any of zero, one, two, three, four, five, six, seven, eight, nine, or 10) amino acid substitutions relative to a wildtype L7Ae protein, wherein the wildtype L7Ae protein comprises the amino acid sequence of any one of SEQ ID NOs:1-10, wherein the L7Ae protein comprises an I or L at position 88, an E or N at position 89, a V at position 90, an N at position 33, an E at position 34, a K at position 37, an R at position 41, and a K at position 79, wherein the position is relative to SEQ ID NO:1.

In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:4, wherein the L7Ae protein comprises an I or L at position 88, an E or N at position 89, or a V at position 90, or combinations thereof, wherein the position is relative to SEQ ID NO:1. In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:4, wherein the L7Ae protein comprises an I or L at position 88, an E or N at position 89, and a V at position 90, wherein the position is relative to SEQ ID NO:1. In some embodiments, the L7Ae protein comprises an I at position 88, an E at position 89, and a V at position 90. In some embodiments, the L7Ae protein comprises an I at position 88, an N at position 89, and a V at position 90. In some embodiments, the L7Ae protein comprises an L at position 88, an N at position 89, and a V at position 90. In some embodiments, the L7Ae protein comprises an L at position 88, an E at position 89, and a V at position 90. In some embodiments, the L7Ae protein does not comprise an arginine (R) at position 90. In some embodiments, the L7Ae protein further comprises one or more (such as any of 2, 3, 4, and 5) of amino acid residues N33, E34, K37, R41, and K79, wherein the position is relative to SEQ ID NO:1. In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:4, wherein the L7Ae protein comprises an I or L at position 88, an E or N at position 89, a V at position 90, an N at position 33, an E at position 34, a K at position 37, an R at position 41, and a K at position 79, wherein the position is relative to SEQ ID NO:1.

In some embodiments, the L7Ae protein comprises no more than 10 (such as any of zero, one, two, three, four, five, six, seven, eight, nine, or 10) amino acid substitutions relative to SEQ ID NO:4, wherein the L7Ae protein comprises an I or L at position 88, an E or N at position 89, or a V at position 90, or combinations thereof, wherein the position is relative to SEQ ID NO:1. In some embodiments, the L7Ae protein comprises no more than 10 (such as any of zero, one, two, three, four, five, six, seven, eight, nine, or 10) amino acid substitutions relative to SEQ ID NO:4, wherein the L7Ae protein comprises an I or L at position 88, an E or N at position 89, and a V at position 90, wherein the position is relative to SEQ ID NO:1. In some embodiments, the L7Ae protein comprises an I at position 88, an E at position 89, and a V at position 90. In some embodiments, the L7Ae protein comprises an I at position 88, an N at position 89, and a V at position 90. In some embodiments, the L7Ae protein comprises an L at position 88, an N at position 89, and a V at position 90. In some embodiments, the L7Ae protein comprises an L at position 88, an E at position 89, and a V at position 90. In some embodiments, the L7Ae protein does not comprise an arginine (R) at position 90. In some embodiments, the L7Ae protein further comprises one or more (such as any of 2, 3, 4, and 5) of amino acid residues N33, E34, K37, R41, and K79, wherein the position is relative to SEQ ID NO:1. In some embodiments, the L7Ae protein comprises no more than 10 (such as any of zero, one, two, three, four, five, six, seven, eight, nine, or 10) amino acid substitutions relative to SEQ ID NO:4, wherein the L7Ae protein comprises an I or L at position 88, an E or N at position 89, a V at position 90, an N at position 33, an E at position 34, a K at position 37, an R at position 41, and a K at position 79, wherein the position is relative to SEQ ID NO:1.

In some embodiments, the L7Ae protein is at least about any of 80, 85, 90, 95, 100, 105, 110, 115, 120, or 125 amino acids long. In some embodiments, the L7Ae protein is about 80-125 amino acids long, including for example about any of 80-90, 90-100, 100-110, 110-120, and 110-125 amino acids long. As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum62 matrix or a PAM250 matrix, and a gap weight of 16, 14, 12, 10, 8, 6, or 4 and a length weight of 1, 2, 3, 4, 5, or 6.

Additionally or alternatively, the protein sequences of the present invention can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the antibody molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. (See www.ncbi.nlm.nih.gov).

For determination of protein sequence identify, the values included are those defined as ‘Identities” by NCBI and do not account for residues that are not conserved but share similar properties (Positives’ as defined by NCBI).

In some embodiments, the L7Ae protein or a variant of a L7Ae protein can be conjugated or linked to a detectable tag or a detectable marker (e.g., a radionuclide, a fluorescent dye, or an MRI-detectable label). In some embodiments, the detectable tag can be an affinity tag. The term “affinity tag,” as used herein, relates to a moiety attached to a polypeptide, which allows the polypeptide to be purified from a biochemical mixture. Affinity tags can consist of amino acid sequences or can include amino acid sequences to which chemical groups are attached by post-translational modifications. Non-limiting examples of affinity tags include His-tag, CBP-tag (CBP: calmodulin-binding protein), CYD-tag (CYD: covalent yet dissociable NorpD peptide), Strep-tag, StrepII-tag, FLAG-tag, HPC-tag (HPC: heavy chain of protein C), GST-tag (GST: glutathione S transferase), Avi-tag, biotinylated tag, Myc-tag, a myc-myc-hexahistidine (mmh) tag 3×FLAG tag, a SUMO tag, and MBP-tag (MBP: maltose-binding protein). Further examples of affinity tags can be found in Kimple et al., Curr Protoc Protein Sci. 2013 Sep. 24; 73: Unit 9.9.

In some embodiments, the detectable tag can be conjugated or linked to the N- and/or C-terminus of a L7Ae protein or a variant of a L7Ae protein. The detectable tag and the affinity tag may also be separated by one or more amino acids. In some embodiments, the detectable tag can be conjugated or linked to the variant via a cleavable element. In the context of the present invention, the term “cleavable element” relates to peptide sequences that are susceptible to cleavage by chemical agents or enzyme means, such as proteases. Proteases may be sequence-specific (e.g., thrombin) or may have limited sequence specificity (e.g., trypsin). Cleavable elements I and II may also be included in the amino acid sequence of a detection tag or polypeptide, particularly where the last amino acid of the detection tag or polypeptide is K or R.

As used herein, the term “conjugate” or “conjugation” or “linked” as used herein refers to the attachment of two or more entities to form one entity. A conjugate encompasses both peptide-small molecule conjugates as well as peptide-protein/peptide conjugates.

The term “fusion polypeptide” or “fusion protein” means a protein created by joining two or more polypeptide sequences together. The fusion polypeptides encompassed in this invention include translation products of a chimeric gene construct that joins the nucleic acid sequences encoding a first polypeptide with the nucleic acid sequence encoding a second polypeptide to form a single open reading frame. In other words, a “fusion polypeptide” or “fusion protein” is a recombinant protein of two or more proteins which are joined by a peptide bond or via several peptides. The fusion protein may also comprise a peptide linker between the two domains.

The term “linker” refers to any means, entity, or moiety used to join two or more entities. A linker can be a covalent linker or a non-covalent linker. Examples of covalent linkers include covalent bonds or a linker moiety covalently attached to one or more of the proteins or domains to be linked. The linker can also be a non-covalent bond, e.g., an organometallic bond through a metal center such as a platinum atom. For covalent linkages, various functionalities can be used, such as amide groups, including carbonic acid derivatives, ethers, esters, including organic and inorganic esters, amino, urethane, urea and the like. To provide for linking, the domains can be modified by oxidation, hydroxylation, substitution, reduction etc., to provide a site for coupling. Methods for conjugation are well known by persons skilled in the art and are encompassed for use in the present invention. Linker moieties include, but are not limited to, chemical linker moieties, or for example, a peptide linker moiety (a linker sequence).

In some embodiments, the linker can be a peptide linker and a non-peptide linker. Examples of the peptide linker may include, without limitation, [S(G)n]m or [S(G)n]mS, where n may be an integer between 1 and 20, and m may be an integer between 1 and 10. For example, the peptide linker can be SG, SGS (SEQ ID NO: 16), SGG (SEQ ID NO: 17), SGGS (SEQ ID NO: 18), SGGG (SEQ ID NO: 19), SGGGS (SEQ ID NO: 20), SGGGG (SEQ ID NO: 21), SGGGGS (SEQ ID NO: 22), SGGGGG (SEQ ID NO: 23), SGGGGGS (SEQ ID NO: 24), SGGGGGG (SEQ ID NO: 25), and SGGSGGGGS (SEQ ID NO: 26).

In some embodiments, the nucleic acid molecule described herein comprises a viral vector. In some embodiments, the viral vector comprises an adeno-associated viral vector, lentiviral vector or adenoviral vector. In some embodiments, the adeno-associated viral vector is selected from AAV1, AAV2, AAV3, AAV4, AAV5, AAV6, AAV7, AAV8, AAV9, AAV10, AAV11, AAV 12, AAV13, AAV rh74, and recombinant subtypes.

A “viral vector” is defined as a recombinantly produced virus or viral particle that comprises a polynucleotide to be delivered into a host cell, either in vivo, ex vivo, or in vitro.

Examples of viral vectors include retroviral vectors, adenovirus vectors, adeno-associated virus vectors, alphavirus vectors, and the like. Infectious tobacco mosaic virus (TMV)-based vectors can be used to produce proteins and have been reported to express Griffithsin in tobacco leaves (O'Keefe et al. (2009) Proc. Nat. Acad. Sci. USA 106(15):6099-6104).

Alphavirus vectors, such as Semliki Forest virus-based vectors and Sindbis virus-based vectors, have also been developed for use in gene therapy and immunotherapy. See, Schlesinger & Dubensky (1999) Curr. Opin. Biotechnol. 5:434-439 and Ying et al. (1999) Nat. Med. 5(7):823-827. In aspects where gene transfer is mediated by a retroviral vector, a vector construct refers to the polynucleotide comprising the retroviral genome or part thereof, and a therapeutic gene. Further details as to modern methods of vectors for use in gene transfer may be found in, for example, Kotterman et al. (2015) Viral Vectors for Gene Therapy: Translational and Clinical Outlook Annual Review of Biomedical Engineering 17.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, a retroviral vector may include a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form, which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses are a relatively well-characterized, homogenous group of viruses, including over 50 serotypes. Adenoviruses do not require integration into the host cell genome. Recombinant adenovirus-derived vectors, particularly those that reduce the potential for recombination and generation of wild-type viruses, have also been constructed. Such vectors are commercially available from sources such as Takara Bio USA (Mountain View, CA), Vector Biolabs (Philadelphia, PA), and Creative Biogene (Shirley, NY). Wild-type adeno-associated virus has high infectivity and specificity integrating into the host cell's genome. See, Wold and Toth (2013) Curr. Gene. Ther. 13(6):421-433, Hermonat & Muzyczka (1984) Proc. Natl. Acad. Sci. USA 81:6466-6470, and Lebkowski et al. (1988) Mol. Cell. Biol. 8:3988,-3996.

Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Gene delivery vehicles also include DNA/liposome complexes, micelles, and targeted viral protein-DNA complexes. Liposomes that also comprise a targeting antibody or fragment thereof can be used in the methods disclosed herein. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins disclosed herein are other non-limiting techniques.

As used herein, the term “viral capsid” or “capsid” refers to the proteinaceous shell or coat of a viral particle. Capsids function to encapsidate, protect, transport, and release into host cell a viral genome. Capsids are generally comprised of oligomeric structural subunits of protein (“capsid proteins”). As used herein, the term “encapsulated” means enclosed within a viral capsid.

As used herein, the term “helper” in reference to a virus or plasmid refers to a virus or plasmid used to provide the additional components necessary for replication and packaging of a viral particle or recombinant viral particle, such as the modified AAV disclosed herein. The components encoded by a helper virus may include any genes required for virion assembly, encapsidation, genome replication, and/or packaging. For example, the helper virus may encode necessary enzymes for the replication of the viral genome. Non-limiting examples of helper viruses and plasmids suitable for use with AAV constructs include pHELP (plasmid), adenovirus (virus), or herpesvirus (virus).

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e., a polynucleotide other than a wild-type AAV genome such as a transgene to be delivered to a mammalian cell), it is typically referred to as an “AAV vector particle” or simply an “AAV vector.” Thus, production of AAV vector particle necessarily includes production of AAV vector, as such a vector is contained within an AAV vector particle.

Adeno-associated virus (AAV) is a replication-deficient parvovirus, the single-stranded DNA genome of which is about 4.7 kb in length, including two 145 nucleotide inverted terminal repeat (ITRs). There are multiple serotypes of AAV. The nucleotide sequences of the genomes of the AAV serotypes are known. For example, the complete genome of AAV1 is provided in GenBank Accession No. NC_002077; the complete genome of AAV2 is provided in GenBank Accession No. NC_001401 and Srivastava et ah, J. Virol., 45: 555-564 (1983); the complete genome of AAV3 is provided in GenBank Accession No. NC_1829; the complete genome of AAV4 is provided in GenBank Accession No. NC_001829; the AAV5 genome is provided in GenBank Accession No. AF085716; the complete genome of AAV6 is provided in GenBank Accession No. NC_00 1862; at least portions of AAV7 and AAV8 genomes are provided in GenBank Accession Nos. AX753246 and AX753249, respectively; the AAV9 genome is provided in Gao et ah, J. Virol., 78: 6381-6388 (2004); the AAV10 genome is provided in Mol. Ther., 13(1): 67-76 (2006); and the AAV1 1 genome is provided in Virology, 330(2): 375-383 (2004). The sequence of the AAV rh.74 genome is provided in U.S. Pat. No. 9,434,928, incorporated herein by reference. Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and pi 9), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter, and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV DNA in the rAAV genomes may be from any AAV serotype for which a recombinant virus can be derived, including, but not limited to, AAV serotypes AAV1, AAV2, AAV3, AAV4, AAV5, AAV6, AAV7, AAV8, AAV9, AAV10, AAV11, AAV 12, AAV13, and AAV rh74.

Production of pseudotyped rAAV is disclosed in, for example, WO 01/83692. Other types of rAAV variants, for example, rAAV with capsid mutations, are also contemplated. See, for example, Marsic et al., Molecular Therapy, 22(11): 1900-1909 (2014). The nucleotide sequences of the genomes of various AAV serotypes are known in the art.

In some embodiments, the influenza virus infection or influenza virus disease is associated with a virus of the orthomycoviridae family, including, but not limited to, an influenza A virus, an influenza B virus, an influenza C virus, and an Isavirus. In some embodiments, the influenza A virus comprises a serotype selected from H1N1, H2N2, H3N1, H3N2, H5N1, H7N7, H1N2, H9N2, H7N2, H7N3, H10N7, H7N9, and H6N1.

Isaviruses are a genus of influenza viruses and members of the family of Orthomyxoviridae. The most studied member of this genus is Infectious Salmon Anemia virus which, like IAV, IBV, and ICV, encodes its genome in eight segments of negative RNA polarity in which segment 7 and 8 undergo splicing. Isaviruses sometimes is also classified as Influenza D viruses (IDV).

As used herein, the terms “subject” and “patient” are used interchangeably irrespective of whether the subject has or is currently undergoing any form of treatment. As used herein, the terms “subject” and “subjects” may refer to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgus monkey, chimpanzee, etc.) and a human) and a non-mammal. The subject may be a human or a non-human. In more exemplary aspects, the mammal is a human. In some embodiments, the subject is a fish, e.g., a salmon fish. In some embodiments, the subject is an avian. In some embodiments, the protein or nucleic acid molecule is administered to the subject prior to the onset of an influenza season.

In some embodiments, the method comprises administrating to the subject a second therapeutic agent or therapy. In some embodiments, the second therapeutic agent comprises an antiviral agent. In some embodiments, the antiviral agent is selected from Oseltamivir, Zanamivir, Amantadine, Rimantadine, Arbidol, Laninamivir, Peramivir, Vitamin D, and an interferon.

In some embodiments, the second therapeutic agent is administered before, after, or concurrently with administering the nucleic acid molecule. In some embodiments, the second therapeutic agent can be formulated in combination with the protein or nucleic acid molecule as described in the same composition or separate compositions.

“Combination” therapy, as used herein, unless otherwise clear from the context, is meant to encompass administration of two or more therapeutic agents in a coordinated fashion and includes, but is not limited to, concurrent dosing. Specifically, combination therapy encompasses both co-administration (e.g., administration of a co-formulation or simultaneous administration of separate therapeutic compositions) and serial or sequential administration, provided that administration of one therapeutic agent is conditioned in some way on the administration of another therapeutic agent. For example, one therapeutic agent may be administered only after a different therapeutic agent has been administered and allowed to act for a prescribed period of time. See, e.g., Kohrt et al. (2011) Blood 117:2423.

As used herein, the term “co-administration” or “co-administered” refers to the administration of at least two agent(s) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary.

### B. METHODS FOR REDUCING INFLUENZA VIRUS REPLICATION

In another aspect, this disclosure also provides a method for reducing influenza virus replication in a subject or a biological sample thereof, e.g., by inhibiting splicing of one or more mRNA segments (e.g., M2 mRNA segment and/or NS2 mRNA segment) in a virus in the orthomyxoviridae family (such as influenza virus). The method comprises: (i) administering to the subject a nucleic acid molecule comprising a polynucleotide encoding an L7Ae protein (e.g., an A. fulgidus L7Ae protein) or a fragment/variant thereof; (ii) administering to the subject an L7Ae protein, e.g., an A. fulgidus L7Ae protein) or a fragment/variant thereof; or (iii) contacting the biological sample with the nucleic acid molecule or the L7Ae protein or fragment/variant thereof. In some embodiments, the nucleic acid molecule or protein is administered prophylactically or therapeutically. In some embodiments, the nucleic acid molecule or protein reduces virus titer in the subject by at least any of 1, 2, 3, or more logs. In some embodiments, the nucleic acid molecule or protein is administered intranasally. In some embodiments, the nucleic acid molecule or protein is administered orally. In some embodiments, the nucleic acid molecule or protein is administered intramuscularly.

In some embodiments, the L7Ae protein comprises an amino acid sequence having at least 75% (e.g., 80%, 85%, 90%, 95%, 96%, 97%, 98%, 99%) identity to any one of SEQ ID NOs: 1- or comprises an amino acid sequence of SEQ ID NOs: 1-10. In some embodiments, the L7Ae protein comprises an amino acid sequence having at least about 75% identify (e.g., at least about any of 80%, 85%, 90%, 95%, 96%, 97%, 98%, 99%, or 100% identity) to SEQ ID NO:1. In some embodiments, the L7Ae protein comprises an amino acid sequence having at least about 75% identify (e.g., at least about any of 80%, 85%, 90%, 95%, 96%, 97%, 98%, 99%, or 100% identity) to SEQ ID NO:4.

In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:4, wherein the L7Ae protein comprises an I or L at position 88, an E or N at position 89, or a V at position 90, or combinations thereof, wherein the position is relative to SEQ ID NO:1. In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:4, wherein the L7Ae protein comprises an I or L at position 88, an E or N at position 89, and a V at position 90, wherein the position is relative to SEQ ID NO:1. In some embodiments, the L7Ae protein comprises an I at position 88, an E at position 89, and a V at position 90. In some embodiments, the L7Ae protein comprises an I at position 88, an N at position 89, and a V at position 90. In some embodiments, the L7Ae protein comprises an L at position 88, an N at position 89, and a V at position 90. In some embodiments, the L7Ae protein comprises an L at position 88, an E at position 89, and a V at position 90. In some embodiments, the L7Ae protein does not comprise an arginine (R) at position 90. In some embodiments, the L7Ae protein further comprises one or more (such as any of 2, 3, 4, and 5) of amino acid residues N33, E34, K37, R41, and K79, wherein the position is relative to SEQ ID NO:1. In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:4, wherein the L7Ae protein comprises an I or L at position 88, an E or N at position 89, a V at position 90, an N at position 33, an E at position 34, a K at position 37, an R at position 41, and a K at position 79, wherein the position is relative to SEQ ID NO:1.

In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:1, wherein the L7Ae protein comprises an I or L at position 88, an E or N at position 89, or a V at position 90, or combinations thereof, wherein the position is relative to SEQ ID NO:1. In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:1, wherein the L7Ae protein comprises an I or L at position 88, an E or N at position 89, and a V at position 90, wherein the position is relative to SEQ ID NO:1. In some embodiments, the L7Ae protein comprises an I at position 88, an E at position 89, and a V at position 90. In some embodiments, the L7Ae protein comprises an I at position 88, an N at position 89, and a V at position 90. In some embodiments, the L7Ae protein comprises an L at position 88, an N at position 89, and a V at position 90. In some embodiments, the L7Ae protein comprises an L at position 88, an E at position 89, and a V at position 90. In some embodiments, the L7Ae protein does not comprise an arginine (R) at position 90. In some embodiments, the L7Ae protein further comprises one or more (such as any of 2, 3, 4, and 5) of amino acid residues N33, E34, K37, R41, and K79, wherein the position is relative to SEQ ID NO:1. In some embodiments, the L7Ae protein comprises an amino acid sequence that is at least about 63% (such as at least about any of 64%, 65%, 70%, 75%, 80%, 85%, 90%, 95%, or 99%) identical to SEQ ID NO:1, wherein the L7Ae protein comprises an I or L at position 88, an E or N at position 89, a V at position 90, an N at position 33, an E at position 34, a K at position 37, an R at position 41, and a K at position 79, wherein the position is relative to SEQ ID NO:1.

In some embodiments, the L7Ae protein is at least about any of 80, 85, 90, 95, 100, 105, 110, 115, 120, or 125 amino acids long. In some embodiments, the L7Ae protein is about 80-125 amino acids long, including, for example, about any of 80-90, 90-100, 100-110, 110-120, and 110-125 amino acids long.

In some embodiments, the polynucleotide comprises a nucleotide sequence having at least 75% (e.g., 80%, 85%, 90%, 95%, 96%, 97%, 98%, 99%) identity to any one of SEQ ID NOs: 11-15, or comprises a nucleotide sequence of SEQ ID NOs: 11-15.

In some embodiments, the L7Ae protein described herein comprises one or more mutations relative to a corresponding wildtype parent protein. For example, in some embodiments, the L7Ae protein comprises a mutation at the K37 and/or K79 position (e.g., K37A and K79A mutations), wherein the position is relative to SEQ ID NO:1.

In some embodiments, the nucleic acid molecule described herein comprises a viral vector. In some embodiments, the viral vector comprises an adeno-associated viral vector, lentiviral vector or adenoviral vector. In some embodiments, the adeno-associated viral vector is selected from AAV1, AAV2, AAV3, AAV4, AAV5, AAV6, AAV7, AAV8, AAV9, AAV10, AAV11, AAV 12, AAV13, AAV rh74, and subtypes thereof.

In some embodiments, when a L7Ae protein or variant/fragment thereof is administered, the protein can be administered via any route known in the art, e.g., via liposome or lipid-based transfection.

In some embodiments, the influenza virus infection or influenza virus disease is associated with a virus in the orthomyxoviridae family, such as an influenza A virus, an influenza B virus, an influenza C virus, and an Isavirus. In some embodiments, the influenza A virus comprises a serotype selected from H1N1, H2N2, H3N1, H3N2, H5N1, H7N7, H1N2, H9N2, H7N2, H7N3, H10N7, H7N9, and H6N1.

The present application in some embodiments provides methods of simultaneously preventing or treating infection with two or more (e.g., any of 2, 3, 4, 5, 6, 7, 8, 9, or more) viruses in the orthomyxoviridae family (also referred herein as influenza virus). Thus, in some embodiments, there is provided a method for simultaneously reducing replication of two or more influenza viruses in a subject or a biological sample thereof, the method comprising: (i) administering to the subject a nucleic acid molecule comprising a polynucleotide encoding an L7Ae protein (e.g., an A. fulgidus L7Ae protein) or a fragment/variant thereof (ii) administering to the subject an L7Ae protein, e.g., an A. fulgidus L7Ae protein) or a fragment/variant thereof; or (iii) contacting the biological sample with the nucleic acid molecule or the L7Ae protein or fragment/variant thereof. In some embodiments, the nucleic acid molecule or protein is administered prophylactically. In some embodiments, the nucleic acid molecule or protein is administered therapeutically, i.e., after the subject has been infected with at least one of the influenza viruses. In some embodiments, the two or more influenza viruses are selected from an influenza A virus, an influenza B virus, an influenza C virus, and Isavirus. In some embodiments, the two or more influenza viruses are influenza A viruses selected from H1N1, H2N2, H3N1, H3N2, H5N1, H7N7, H1N2, H9N2, H7N2, H7N3, H10N7, H7N9, and H6N1 influenza A viruses. In some embodiments, the nucleic acid molecule or protein administered to the subject elicits no (or substantially no) immune response in the subject. In some embodiments, the nucleic acid molecule or protein has no impact (or substantially no impact) on the host transcriptome and/or proteome. In some embodiments, the nucleic acid molecule or protein reduces virus titer by at least any of 1, 2, 3, or more logs. In some embodiments, the nucleic acid molecule or protein is administered intranasally. In some embodiments, the nucleic acid molecule or protein is administered orally. In some embodiments, the nucleic acid molecule or protein is administered intramuscularly.

In some embodiments, there is provided a method for preventing or treating influenza virus infections in a subject or a biological sample thereof, the method comprising: (i) administering to the subject a nucleic acid molecule comprising a polynucleotide encoding an L7Ae protein (e.g., an A. fulgidus L7Ae protein) or a fragment/variant thereof (ii) administering to the subject an L7Ae protein, e.g., an A. fulgidus L7Ae protein) or a fragment/variant thereof; or (iii) contacting the biological sample with the nucleic acid molecule or the L7Ae protein or fragment/variant thereof. In some embodiments, the method is broad spectrum, i.e., useful for treating the influenza virus regardless of the subtypes or serotypes. In some embodiments, the method is for preventing or treating infections with an influenza A virus, an influenza B virus, an influenza C virus, and/or an Isavirus. In some embodiments, the method is for preventing or treating infections with H1N1, H2N2, H3N1, H3N2, H5N1, H7N7, H1N2, H9N2, H7N2, H7N3, H10N7, H7N9, and/or H6N1 influenza A viruses. In some embodiments, the nucleic acid molecule or protein administered to the subject elicits no (or substantially no) immune response in the subject. In some embodiments, the nucleic acid molecule or protein has no impact (or substantially no impact) on the host transcriptome and/or proteome. In some embodiments, the nucleic acid molecule or protein reduces virus titer by at least any of 1, 2, 3, or more logs. In some embodiments, the nucleic acid molecule or protein is administered intranasally. In some embodiments, the nucleic acid molecule or protein is administered orally. In some embodiments, the nucleic acid molecule or protein is administered intramuscularly.

In some embodiments, there is provided a method for preventing or treating infection of a virus (such as any virus) in the orthomyxoviridae family in a subject or a biological sample thereof, the method comprising: (i) administering to the subject a nucleic acid molecule comprising a polynucleotide encoding an L7Ae protein (e.g., an A. fulgidus L7Ae protein) or a fragment/variant thereof; (ii) administering to the subject an L7Ae protein, e.g., an A. fulgidus L7Ae protein) or a fragment/variant thereof; or (iii) contacting the biological sample with the nucleic acid molecule or the L7Ae protein or fragment/variant thereof. In some embodiments, the nucleic acid or protein molecule administered to the subject elicits no (or substantially no) immune response in the subject. In some embodiments, the nucleic acid molecule or protein administered to the subject elicits no (or substantially no) immune response in the subject. In some embodiments, the nucleic acid molecule or protein has no impact (or substantially no impact) on the host transcriptome and/or proteome. In some embodiments, the nucleic acid molecule or protein reduces virus titer by at least any of 1, 2, 3, or more logs. In some embodiments, the nucleic acid molecule or protein is administered intranasally. In some embodiments, the nucleic acid molecule or protein is administered orally. In some embodiments, the nucleic acid molecule or protein is administered intramuscularly.

In some embodiments, the method is for preventing or treating an influenza virus infection or influenza virus disease associated with an influenza A virus, an influenza B virus, or an influenza C virus in a mammal, such as a human individual. In some embodiments, the influenza A virus comprises a serotype selected from H1N1, H2N2, H3N1, H3N2, H5N1, H7N7, H1N2, H9N2, H7N2, H7N3, H10N7, H7N9, and H6N1.

In some embodiments, the method is for preventing or treating an influenza virus infection or influenza virus disease associated with an Isavirus in a fish (such as a salmon fish).

In some embodiments, the method is for preventing or treating an influenza virus infection or influenza virus disease associated with an avian flu, such as any of H1N1, H2N2, H3N1, H3N2, H5N1, H7N7, H1N2, H9N2, H7N2, H7N3, H10N7, H7N9, or H6N1 virus, in an avian.

In another aspect, this disclosure further provides a method for inhibiting splicing of one or more influenza virus mRNA segments in a subject or a biological sample thereof. The method comprises: (i) administering to the subject a nucleic acid molecule comprising a polynucleotide encoding an L7Ae protein (e.g., an A. fulgidus L7Ae protein) or a fragment/variant thereof; (ii) administering to the subject an L7Ae protein, e.g., an A. fulgidus L7Ae protein) or a fragment/variant thereof; or (iii) contacting the biological sample with the nucleic acid molecule or the L7Ae protein or fragment/variant thereof. In some embodiments, the one or more influenza virus mRNA segments comprise M2 mRNA segment, NS2 mRNA segment, or both. In some embodiments, the influenza virus mRNA segments are present in one or more (e.g., 2 or 3) influenza viruses selected from an influenza A virus, an influenza B virus, and influenza C virus. In some embodiments, the influenza virus mRNA segments are present in one or more (e.g., 2, 3 or 4,) influenza viruses selected from an influenza A virus, an influenza B virus, an influenza C virus, and an Isavirus. In some embodiments, the influenza virus mRNA segments are present in one or more (e.g., 2, 3, 4, 5, 6, 7, 8, 9, 10, 11, 12, or more) influenza viruses selected from H1N1, H2N2, H3N1, H3N2, H5N1, H7N7, H1N2, H9N2, H7N2, H7N3, H10N7, H7N9, and H6N1 influenza A viruses. In some embodiments, the influenza virus mRNA is present in all viruses in the orthomyxoviridae family.

The influenza A and B virus genomes each comprise eight negative-sense, single-stranded viral RNA (vRNA) segments (PB2, PB1, PA, HA, NP, NA, M, and NS), while the influenza C virus has a seven-segment genome (PB2, PB1, PA, NP, HEF, M, and NS). The eight segments of influenza A and B viruses (and the seven segments of influenza C virus) are numbered in order of decreasing length. In influenza A and B viruses, segments 1, 3, 4, and 5 encode just one protein per segment: the PB2, PA, HA, and NP proteins. All influenza viruses encode the polymerase subunit PB1 on segment 2; in some strains of influenza A virus, this segment also codes for the accessory protein PB1-F2, a small, 87-amino acid protein with pro-apoptotic activity, in a +1 alternate reading frame. No analogue to PB1-F2 has been identified in influenza B or C viruses. Conversely, segment 6 of the influenza A virus encodes only the NA protein, while that of influenza B virus encodes both the NA protein and, in a −1 alternate reading frame, the NB matrix protein, which is an integral membrane protein corresponding to the influenza A virus M2 protein. Segment 7 of both influenza A and B viruses code for the M1 matrix protein. In the influenza A genome, the M2 ion channel is also expressed from segment 7 by RNA splicing, while the influenza B virus encodes its BM2 membrane protein in a +2 alternate reading frame. Finally, both influenza A and B viruses possess a single RNA segment, segment 8, from which they express the interferon-antagonist NS1 protein and, by mRNA splicing, the NEP/NS2, which is involved in viral RNP export from the host cell nucleus. The genomic organization of influenza C viruses is generally similar to that of influenza A and B viruses; however, the HEF protein of influenza C replaces the HA and NA proteins, and thus the influenza C virus genome has one fewer segment than that of influenza A or B viruses.

The dose of the proteins or nucleic acid molecules to be administered to the subject depends on the subject. When administered nasally, the protein can be administered at a dose of about 10 μg/kg to about 10 mg/kg, such as about 40 mg/kg to about 100 mg/kg, and the nucleic acid molecule can be administered at a dose of about 10⁹ to about 10¹² genomic copies per administration, such as about 10¹¹ genomic copies per administration. The protein or nucleic acid molecules can be administered a single time, or multiple times.

### C. VECTORS, CELLS, AND COMPOSITIONS

In yet another aspect, this disclosure additional provides a vector comprising a polynucleotide encoding an L7Ae protein (e.g., an A. fulgidus L7Ae protein) or a fragment/variant thereof. In some embodiments, the L7Ae protein comprises an amino acid sequence having at least 75% (e.g., 80%, 85%, 90%, 95%, 96%, 97%, 98%, 99%) identity to any one of SEQ ID NOs: 1-10, or comprises an amino acid sequence of SEQ ID NOs: 1-10. In some embodiments, the polynucleotide comprises a nucleotide sequence having at least 75% (e.g., 80%, 85%, 90%, 95%, 96%, 97%, 98%, 99%) identity to any one of SEQ ID NOs: 11-15, or comprises a nucleotide sequence of SEQ ID NOs: 11-15.

In some embodiments, the vector is a viral vector. In some embodiments, the viral vector is an adeno-associated viral vector, lentiviral vector or adenoviral vector. In some embodiments, the adeno-associated viral vector is selected from AAV1, AAV2, AAV3, AAV4, AAV5, AAV6, AAV7, AAV8, AAV9, AAV10, AAV11, AAV 12, AAV13, AAV rh74, and recombinant subtypes thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode mutant polypeptides or immunoconjugates of the invention or fragments thereof.

Also provided in this disclosure is a host cell comprising the vector described hererin. The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progenies that have the same function or biological activity as screened or selected for in the originally transformed cell are included herein.

In some embodiments, the vector or the host cell, can be incorporated into pharmaceutical compositions suitable for administration. As used herein, the term “composition” or “pharmaceutical composition” refers to a mixture of at least one component useful within the invention with other components, such as carriers, stabilizers, diluents, dispersing agents, suspending agents, thickening agents, and/or excipients. The pharmaceutical composition facilitates administration of one or more components of the invention to an organism.

The pharmaceutical compositions generally comprise a substantially purified nucleic acid molecule or protein described herein and a pharmaceutically acceptable carrier in a form suitable for administration to a subject. Pharmaceutically-acceptable carriers are determined in part by the particular composition being administered, as well as by the particular method used to administer the composition. The pharmaceutical compositions are generally formulated as sterile, substantially isotonic and in full compliance with all Good Manufacturing Practice (GMP) regulations of the U.S. Food and Drug Administration.

The terms “pharmaceutically acceptable,” “physiologically tolerable,” as referred to compositions, carriers, diluents, and reagents, are used interchangeably and include materials are capable of administration to or upon a subject without the production of undesirable physiological effects to the degree that would prohibit administration of the composition. For example, “pharmaceutically-acceptable excipient” includes an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

Examples of such carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the nucleic acid molecule, use thereof in the compositions is contemplated. Supplementary active compounds can also be incorporated into the compositions.

A pharmaceutical composition is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfate; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate-buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. In many cases, it will be preferable to include isotonic compounds, e.g., sugars, polyalcohols such as mannitol, sorbitol, sodium chloride in the composition. Prolonged absorption of the injectable compositions can be brought about by including in the composition a compound which delays absorption, e.g., aluminum monostearate and gelatin.

Sterile injectable solutions can be prepared by incorporating the nucleic acid molecule or protein in the required amount in an appropriate solvent with one or a combination of ingredients enumerated above, as required. Generally, dispersions are prepared by incorporating the nucleic acid molecule or protein into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The nucleic acid molecule or protein can be administered in the form of a depot injection or implant preparation, which can be formulated in such a manner as to permit a sustained or pulsatile release of the active ingredient.

Systemic administration can also be by transmucosal or transdermal means. For transmucosal or transdermal administration, penetrants appropriate to the barrier to be permeated are used in the formulation. Such penetrants are generally known in the art, and include, e.g., for transmucosal administration, detergents, bile salts, and fusidic acid derivatives. For transdermal administration, the nucleic acid molecule or protein is formulated into ointments, salves, gels, or creams as generally known in the art.

In some embodiments, the nucleic acid molecule or protein is prepared with carriers that will protect the nucleic acid molecule or protein against rapid elimination from the body, such as a controlled release formulation, including implants and microencapsulated delivery systems. Biodegradable, biocompatible polymers can be used, such as ethylene-vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically-acceptable carriers.

In some embodiments, the composition includes the nucleic acid or protein molecule as described herein and optionally a cryo-protectant (e.g., glycerol, DMSO, PEG).

Also within the scope of this disclosure is a kit comprising the vector, the host cell or the composition described herein. The kit may further include instructions for administrating the nucleic acid molecule or the composition and optionally an adjuvant. The kit optionally includes a device suitable for administration of the composition, e.g., a syringe or other suitable delivery device. The device can be provided pre-loaded with one or both of the agents or can be empty, but suitable for loading.

### D. DEFINITIONS

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

A polynucleotide disclosed herein can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell, such as an extrachromosomal replicon (e.g., a plasmid) or a nuclear or mitochondrial chromosome. A number of vectors are known to be capable of mediating transfer of genes to mammalian cells, as is known in the art and described herein.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively, the proteins produced may act as toxins under similar circumstances. “Plasmids” used in genetic engineering are called “plasmid vectors.” Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene.

The term “amino acid sequence” refers to an amino acid sequence of a protein molecule, “amino acid sequence” and like terms, such as “polypeptide” or “protein” are not meant to limit the amino acid sequence to the complete, native amino acid sequence associated with the recited protein molecule. Furthermore, an “amino acid sequence” can be deduced from the nucleic acid sequence encoding the protein.

The term “gene” refers to a nucleic acid (e.g., DNA or RNA) sequence that comprises coding sequences necessary for the production of an RNA, or a polypeptide or its precursor (e.g., proinsulin). A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends for a distance of about 1 kb on either end such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns, therefore, are absent in the messenger RNA (mRNA) transcript. The mRNA functions during translation to specify the sequence or order of amino acids in a nascent polypeptide.

The term “recombinant” when made in reference to a nucleic acid molecule refers to a nucleic acid molecule that is comprised of segments of nucleic acid joined together by means of molecular biological techniques. The term “recombinant,” when made in reference to a protein or a polypeptide, refers to a protein molecule which is expressed using a recombinant nucleic acid molecule.

The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The terms “culture,” “culturing,” “grow,” “growing,” “maintain,” “maintaining,” “expand,” “expanding,” etc., when referring to cell culture itself or the process of culturing, can be used interchangeably to mean that a cell (e.g., primary cell) is maintained outside its normal environment under controlled conditions, e.g., under conditions suitable for survival. Cultured cells are allowed to survive, and culturing can result in cell growth, stasis, differentiation or division. The term does not imply that all cells in the culture survive, grow, or divide, as some may naturally die or senesce. Cells are typically cultured in media, which can be changed during the course of the culture.

As used herein, the term “contacting,” when used in reference to any set of components, includes any process whereby the components to be contacted are mixed into the same mixture (for example, are added into the same compartment or solution), and does not necessarily require actual physical contact between the recited components. The recited components can be contacted in any order or any combination (or sub-combination) and can include situations where one or some of the recited components are subsequently removed from the mixture, optionally prior to addition of other recited components. For example, “contacting A with B and C” includes any and all of the following situations: (i) A is mixed with C, then B is added to the mixture; (ii) A and B are mixed into a mixture; B is removed from the mixture, and then C is added to the mixture; and (iii) A is added to a mixture of B and C. “Contacting” a target nucleic acid or a cell with one or more reaction components includes any or all of the following situations: (i) the target or cell is contacted with a first component of a reaction mixture to create a mixture; then other components of the reaction mixture are added in any order or combination to the mixture; and (ii) the reaction mixture is fully formed prior to mixture with the target or cell.

The term “disease” as used herein is intended to be generally synonymous and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The terms “decrease,” “reduced,” “reduction,” “decrease,” or “inhibit” are all used herein generally to mean a decrease by a statistically significant amount. However, for avoidance of doubt, “reduced,” “reduction” or “decrease” or “inhibit” means a decrease by at least 10% as compared to a reference level, for example, a decrease by at least about 20%, or at least about 30%, or at least about 40%, or at least about 50%, or at least about 60%, or at least about 70%, or at least about 80%, or at least about 90% or up to and including a 100% decrease (e.g., absent level as compared to a reference sample), or any decrease between 10-100% as compared to a reference level.

The terms “increased,” “increase” or “enhance” or “activate” are all used herein to generally mean an increase by a statically significant amount; for the avoidance of any doubt, the terms “increased,” “increase” or “enhance” or “activate” means an increase of at least 10% as compared to a reference level, for example, an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “effective amount,” “effective dose,” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A “prophylactically effective amount” or a “prophylactically effective dosage” of a drug is an amount of the drug that, when administered alone or in combination with another therapeutic agent to a subject at risk of developing a disease or of suffering a recurrence of disease, inhibits the development or recurrence of the disease. The ability of a therapeutic or prophylactic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

Doses are often expressed in relation to bodyweight. Thus, a dose which is expressed as [g, mg, or other unit]/kg (or g, mg etc.) usually refers to [g, mg, or other unit] “per kg (or g, mg etc.) bodyweight,” even if the term “bodyweight” is not explicitly mentioned.

The term “agent” is used herein to denote a chemical compound, a mixture of chemical compounds, a biological macromolecule (such as a nucleic acid, an antibody, a protein or portion thereof, e.g., a peptide), or an extract made from biological materials such as bacteria, plants, fungi, or animal (particularly mammalian) cells or tissues. The activity of such agents may render it suitable as a “therapeutic agent,” which is a biologically, physiologically, or pharmacologically active substance (or substances) that acts locally or systemically in a subject.

The terms “therapeutic agent,” “therapeutic capable agent,” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, the term “pharmaceutical grade” means that certain specified biologically active and/or inactive components in the drug must be within certain specified absolute and/or relative concentration, purity and/or toxicity limits and/or that the components must exhibit certain activity levels, as measured by a given bioactivity assay. Further, a “pharmaceutical grade compound” includes any active or inactive drug, biologic or reagent, for which a chemical purity standard has been established by a recognized national or regional pharmacopeia (e.g., the U.S. Pharmacopeia (USP), British Pharmacopeia (BP), National Formulary (NF), European Pharmacopoeia (EP), Japanese Pharmacopeia (JP), etc.). Pharmaceutical grade further incorporates suitability for administration by means including topical, ocular, parenteral, nasal, pulmonary tract, mucosal, vaginal, rectal, intravenous, and the like.

“Sample,” “test sample,” and “patient sample” may be used interchangeably herein. The sample can be a sample of, serum, urine plasma, amniotic fluid, cerebrospinal fluid, cells (e.g., antibody-producing cells) or tissue. Such a sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art. The terms “sample” and “biological sample” as used herein generally refer to a biological material being tested for and/or suspected of containing an analyte of interest, such as antibodies. The sample may be any tissue sample from the subject. The sample may comprise protein from the subject.

Any cell type, tissue, or bodily fluid may be utilized to obtain a sample. Such cell types, tissues, and fluid may include sections of tissues such as biopsy and autopsy samples, frozen sections taken for histologic purposes, blood (such as whole blood), plasma, serum, sputum, stool, tears, mucus, saliva, hair, skin, red blood cells, platelets, interstitial fluid, ocular lens fluid, cerebral spinal fluid, sweat, nasal fluid, synovial fluid, menses, amniotic fluid, semen, etc. Cell types and tissues may also include lymph fluid, ascetic fluid, gynecological fluid, urine, peritoneal fluid, cerebrospinal fluid, a fluid collected by vaginal rinsing, or a fluid collected by vaginal flushing. A tissue or cell type may be provided by removing a sample of cells from an animal, but can also be accomplished by using previously isolated cells (e.g., isolated by another person, at another time, and/or for another purpose). Archival tissues, such as those having treatment or outcome history, may also be used. Protein purification may not be necessary.

Methods well known in the art for collecting, handling, and processing urine, blood, serum, and plasma, and other body fluids, can be used in the practice of the present disclosure, for instance. The test sample can comprise further moieties in addition to the analyte of interest, such as antibodies, antigens, haptens, hormones, drugs, enzymes, receptors, proteins, peptides, polypeptides, oligonucleotides or polynucleotides. For example, the sample can be a whole blood sample obtained from a subject. Even in cases where pretreatment is not necessary, pretreatment optionally can be done for mere convenience (e.g., as part of a regimen on a commercial platform). The sample may be used directly as obtained from the subject or following a pretreatment to modify a characteristic of the sample. Pretreatment may include extraction, concentration, inactivation of interfering components, and/or the addition of reagents.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a non-human animal.

It is noted here that, as used in this specification and the appended claims, the singular forms “a,” “an,” and “the” include plural reference unless the context clearly dictates otherwise.

The terms “including,” “comprising,” “containing,” or “having” and variations thereof are meant to encompass the items listed thereafter and equivalents thereof as well as additional subject matter unless otherwise noted.

The phrases “in one embodiment,” “in various embodiments,” “in some embodiments,” and the like are used repeatedly. Such phrases do not necessarily refer to the same embodiment, but they may unless the context dictates otherwise.

The terms “and/or” or “/” means any one of the items, any combination of the items, or all of the items with which this term is associated.

The word “substantially” does not exclude “completely,” e.g., a composition which is “substantially free” from Y may be completely free from Y. Where necessary, the word “substantially” may be omitted from the definition of the invention.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise.

In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### E. EXAMPLES

### Example 1

This example describes the materials and methods to be used in the subsequent examples.

Cell Culture and Transfection.

Human embryonic kidney 293T (HEK293T) cells (ATCC, CRL-3216), Human adenocarcinomic alveolar basal epithelial (A549) cells (ATCC, CCL-185), Madin-Darby Canine Kidney (MDCK) cells (ATCC, CCL-34), and African green monkey kidney epithelial Vero E6 cells (ATCC, CRL-1586) were maintained at 37° C. and 5% CO₂ in Dulbecco's modified Eagle's Medium (DMEM, Gibco) supplemented with 10% Fetal Bovine Serum (FBS) and 1% penicillin-streptomycin. All cells were routinely screened for mycoplasma. All plasmid transfection was performed in OptiMem (Life technologies) using lipofectamine 2000 (Invitrogen).

Dox-Inducible Gene Expressing Cell Lines.

Lentiviral vectors encoding L7Ae-2A-YFP were generated using the pTRE3G-BFP2-PuroR backbone vector. Third-generation VSV-G pseudotype HIV-I lentiviruses were produced by transfection of HEK293T cells. At 48 hours post-transfection, supernatants from transfected cells were harvested and added to MDCK or A549 cells with polybrene (Santa Cruz Biotechnology) as described elsewhere (Eggenberger, J., et al. Proc Natl Acad Sci USA 116, 1384-1393 (2019)). After 48 hours, cells were re-seeded and incubated in complete media containing 0.5 μg/ml puromycin to select lentivirus infected cells. After two weeks, each colony formed by a single cell was picked. For dox-induction experiments, 0.5 μg/ml doxycycline (Sigma, D9891) was replaced at 24 hours with fresh media.

Virus rescue and growth kinetics.

Plasmid-Based Reverse Genetics for Influenza Virus Generation was Performed as Previously described (Fodor, E. et al. J Virol 73, 9679-9682 (1999); Hoffmann, E., et al. Proc Natl Acad Sci USA 97, 6108-6113 (2000)). For growth kinetics, MDCK or MDCK-dox inducible L7Ae-2A-YFP cells were infected with influenza virus at each MOI; A/Puerto Rico/08/34 (H1N1) (MOI=0.001), modified A/Puerto Rico/08/34 (H1N1) (MOI=0.01), A/Texas/36/91 (H1N1) (MOI=0.01), A/California/04/2009 (H1N1pdm09) (MOI=0.01), A/Netherlands/602/2009 (H1N1pdm09) (MOI=0.01), A/Panama/2007/99 (MOI=0.01), live-attenuated A/Viet Nam/1203/2004 (H5N1) (M01=0.01), or influenza B/Yamagata/16/88 virus (MOI=0.01). After incubation for 1 hour, inoculum was replaced with EMEM containing 0.3% BSA and 1 μg/ml TPCK-trypsin, and cells were incubated at 37° C. for influenza A virus, 33° C. for influenza B virus. Cells were infected with VSV-GFP at a MOI=0.01 and incubated at 37° C. Virus titers of influenza virus or VSV in the cell culture supernatants that were collected at the indicated time points were examined by means of plaque assay in MDCK cells or Vero cells, respectively.

In Vivo Infections.

WT female Balb/c mice were purchased at 4-6 weeks of age from Jackson Laboratories (Bar Harbor, ME). Mice were anesthetized by intraperitoneal injection with xylene and ketamine and administered an AAV9 vector expressing YFP or L7Ae-2A-YFP. AV9 vectors flanked with AAV2 inverted terminal repeats contained either the YFP gene fused or L7Ae-2A-YFP under the transcriptional control of the cytomegalovirus-enhanced chicken β-actin promoter. AAV vectors were commercially produced by Vector Biolabs. Mice were inoculated intranasally with 10¹¹ genome copies of either vector in a final volume of 25 μl one day prior to A/PR/8/34 infection (10LD50) (Huang, L. & Lilley, D. M. RNA 19, 1703-1710 (2013)). Mock-treated animals received PBS only. Each cohort was comprised of 6 animals. Animals were monitored daily and weighed as a proxy for morbidity. All animal experiments were performed as biological triplicates according to protocols approved by the Institutional Animal Care and Use Committee (IACUC) and Institutional Biosafety Committee of the Icahn School of Medicine at Mount Sinai (NY, USA). Mice were randomly assigned to the different experimental groups.

Western Blotting.

Cells were lysed in a SDS sample buffer. Whole cell lysates were separated on 4-15% Mini protean TGX gels (BioRad) and transferred onto a nitrocellulose membrane (BioRad). Proteins were detected using mouse monoclonal anti-FLAG (Sigma, HT103), mouse monoclonal anti-Myc (Cell Signaling, 9B11), mouse monoclonal anti-NP (Kerafast, EMS010), mouse monoclonal anti-NA (4A5), mouse monoclonal anti-M1/M2 (Kerafast, E10), mouse monoclonal anti-NS1 (ISMMS hybridoma Center, 1A7), rabbit polyclonal anti-NS2 (Genescript, A01499), rabbit monoclonal anti-GAPDH (Sigma, G9545) (Wohlbold, T. J. et al. mBio 6, e02556 (2015).). Peroxidase-conjugated anti-mouse or -rabbit secondary antibody (GE Healthcare) was used. For antibody detection, Immobilon Western Chemiluminescent HRP substrate (Millipore) was used.

RNA Analysis.

Total RNA was isolated from respective samples by TRIzol (Thermo Fisher) extractions and DNase-treated using DNase I recombinant (Roche). RNA-seq libraries of polyadenylated RNA were prepared using Truseq stranded mRNA Library Prep kit (Illumina) according to the manufacturer's instructions. cDNA libraries were sequenced using an Illumine NextSeq500 platform and analyzed using Bowtie2 and deSEQ2. For quantitative RT-PCR, RNA was reverse transcribed into cDNA using oligo-dT primers with Superscript II Reverse transcriptase (Thermo Fisher). Real-time PCR was performed using primers specific for tubulin, M1, M2, NS1 or NS2 with KAPA SYBR Fast qPCR Master Mix (KAPA Biosystems) on LightCycler 480 Instrument II (Roche). Delta-delta-cycle threshold was determined relative to non-dox-treated infected samples.

Selection of Escape Mutant Viruses.

L7Ae-mediated escape mutants were selected by passaging virus on MDCK-dox-inducible L7Ae-2A-YFP cells with dox-treatment. 48 hours post-infection, viruses-containing supernatant was harvested and diluted to 1:1000. Diluted viruses were then inoculated into naïve MDCK-dox-inducible L7Ae-2A-YFP cells in the presence of doxycycline. After passage 20, viruses-containing supernatants were collected and used for plaque assay, from which twenty single plaques were picked and amplified on MDCK cells. Their M and NS genes were analyzed by RT-PCR and sequenced.

Determination of Viral Fitness.

Wild-type and NS-G60A mutant viruses were mixed at an equal ratio on the basis of their virus titers. Virus mixtures were inoculated into MDCK-dox-inducible L7Ae-2A-YFP cells with or without dox-treatment. 48 hours post-infection, supernatants from each infected cell were collected as passage 1 samples. Passage 1 samples were diluted to 1:1000 and inoculated into MDCK-dox-inducible L7Ae-2A-YFP cells with or without dox-treatment again. 48 hours post-infection, supernatants from infected cells were collected and further passaged. Total RNA was isolated from each passage and reverse transcribed into cDNA using influenza virus-specific primers with Superscript II Reverse transcriptase (Thermo Fisher). Fragment of NS segment was amplified by PCR and purified using a QIAquick Gel Extraction Kit (QIAGEN). Purified DNA fragments were analyzed by amplicon sequencing (Genewiz). For each passage, biological triplicates were analyzed.

RIP-Seq.

HEK293T cells were transfected with the plasmid encoding epitope-tagged L7Ae. After 24 h, cells were infected with A/Puerto Rico/08/34 (H1N1) at a MOI=1. At 24 hours-post-infection, cells were lysed in the lysis buffer containing 50 mM Tris-HCl (pH 7.5), 150 mM NaCl, 0.5% Nonidet P-40, RNaseOUT (Thermo Fisher), and protease inhibitor Complete Mini cocktail (Roche) for 1 hour at 4° C. After centrifugation to remove cellular debris, the supernatants were incubated with anti-FLAG M2 magnetic beads (Sigma) overnight at 4° C. The magnetic beads were washed three times with the lysis buffer and then washed twice with wash buffer (50 mM Tris-HCl, pH 7.5, and 150 mM NaCl). RNA or proteins bound to the magnetic beads were released by adding TRIzol or SDS sample buffer. RNA was isolated for RNA-Seq, as described. Eluted proteins were analyzed by western blotting as described.

### Example 2

This disclosure is based, in part, on a discovery that a family of evolutionary conserved Archaea RNA binding proteins belonging to the L30 family unexpectedly blocked influenza virus-mediated splicing regardless of the influenza virus strain. Moreover, it was further demonstrated in this disclosure that this activity, while inhibiting every tested strain of Influenza A and B viruses, had no significant impact on the host, nor could viral escape of L30-based targeting be observed. This disclosure also demonstrated that delivery of a prototype member of the L30 family was sufficient to protect animals from a lethal challenge of virus and propose that these findings represent a bone fide universal therapy for seasonal or pandemic influenza threats of the future.

The cellular response to virus infection is a complex process that has been shaped over evolutionary time (tenOever, B. R. Cell Host Microbe 19, 142-149 (2016)). Despite this, these defense systems retain some basic general principles that undoubtedly reflect some of the more successful strategies at inhibiting virus replication. For example, it was found that RNA binding proteins, notably RNAse III endonucleases, provide a thread of continuity to the antiviral defenses of all life on this planet (Aguado, L. C. et al. Nature 547, 114-117 (2017)). RNAse III in bacteria participates in the maturation of small RNAs for the CRISPR Type II system. It is required to process small RNAs in the RNA interference (RNAi) defense. This family of proteins also shows homology to the pattern recognition receptors of our interferon system (Deltcheva, E. et al. Nature 471, 602-607 (2011); Bernstein, E., et al. Nature 409, 363-366 (2001); Luo, D. et al. Cell 147, 409-422 (2011)).

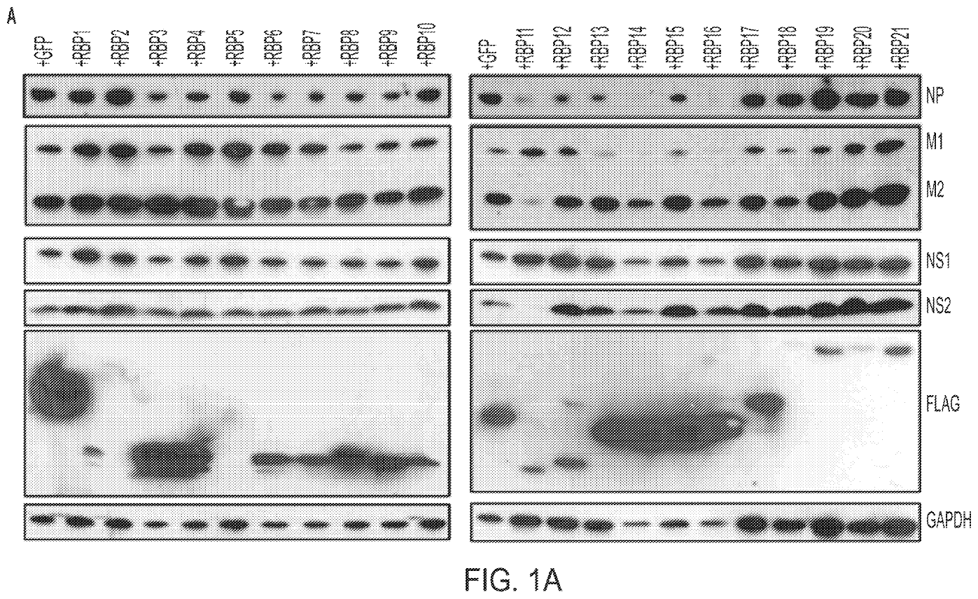

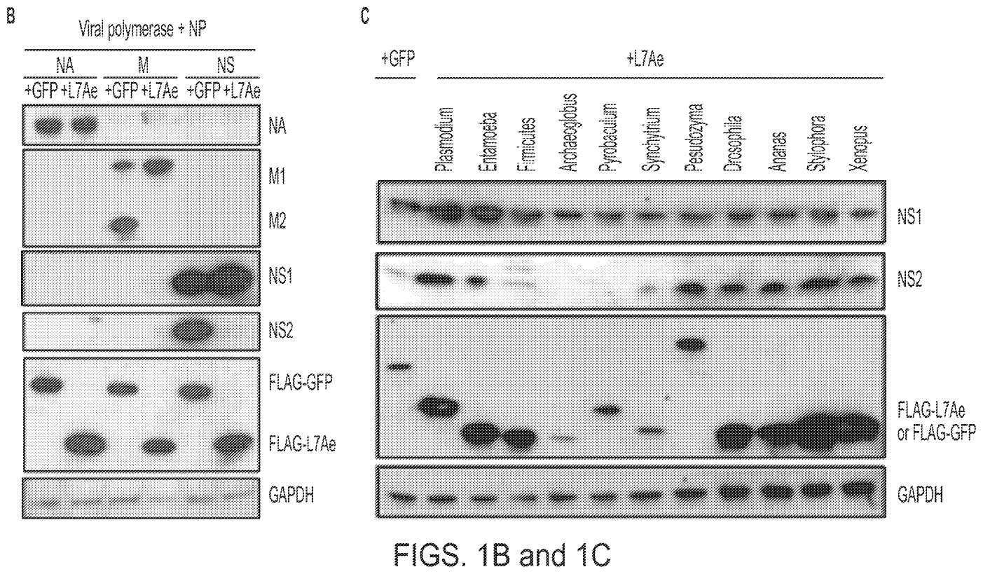

Given these findings, a survey of a library of RNA binding proteins from Archaea was conducted to ascertain whether they showed any antiviral activity against Influenza A virus (herein denoted IAV). To this end, 21 codon-optimized RNA binding proteins from a diverse array of archaea were synthesized (FIG. 1A and Table 1). These constructs were exogenously expressed in fibroblasts and subsequently infected with IAV (H1N1, A/Puerto Rico/08/1934) to test their capacity to interfere with aspects of virus replication. In an effort to capture the full breadth of virus biology, segment eight was examined as it undergoes both canonical and non-canonical processes that ultimately control the viral replication cycle (Chua, M. A., et al. Cell Rep 3, 23-29 (2013)). These data demonstrated that RBP11, RBP14, and RBP16 all successfully reduced virus levels as determined by a diminished signal for nucleoprotein (NP), matrix 1 (M1), matrix 2 (M2), non-structural protein 1 (NS1), and/or non-structural protein 2 (NS2) (FIG. 1A). It is of note that while RBP14 and RBP16 both encode RNAseIII nucleases and thus presumably inhibit infection by engaging viral RNA hairpins as described elsewhere, RBP11 exhibited a distinct phenotype as it appears to preferentially block production of the two spliced products of IAV, M2 and NS2 (also known as the nuclear export protein or NEP) (FIG. 1A) (Aguado, L. C. et al. Nature 547, 114-117 (2017); O'Neill, R. E., et al. EMBO J 17, 288-296 (1998)).

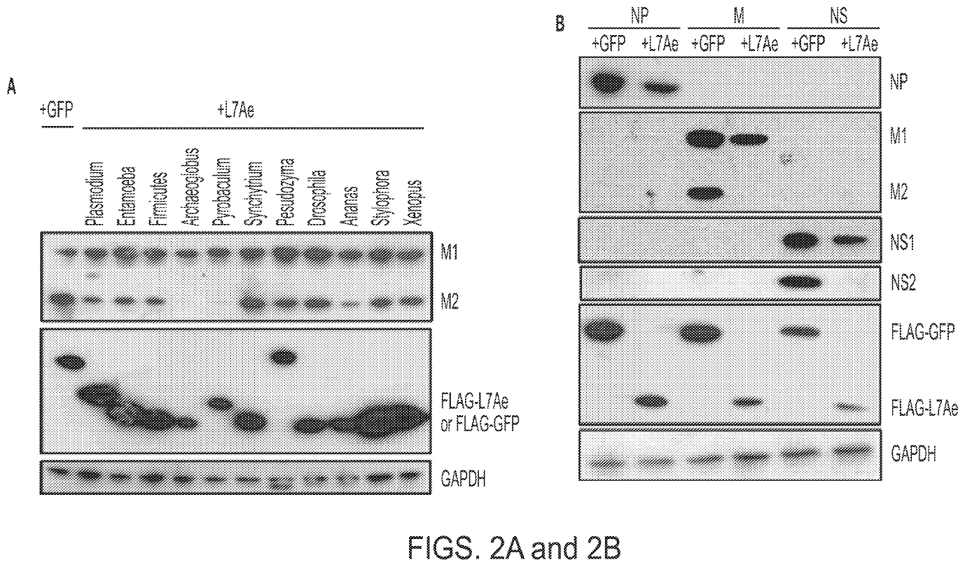

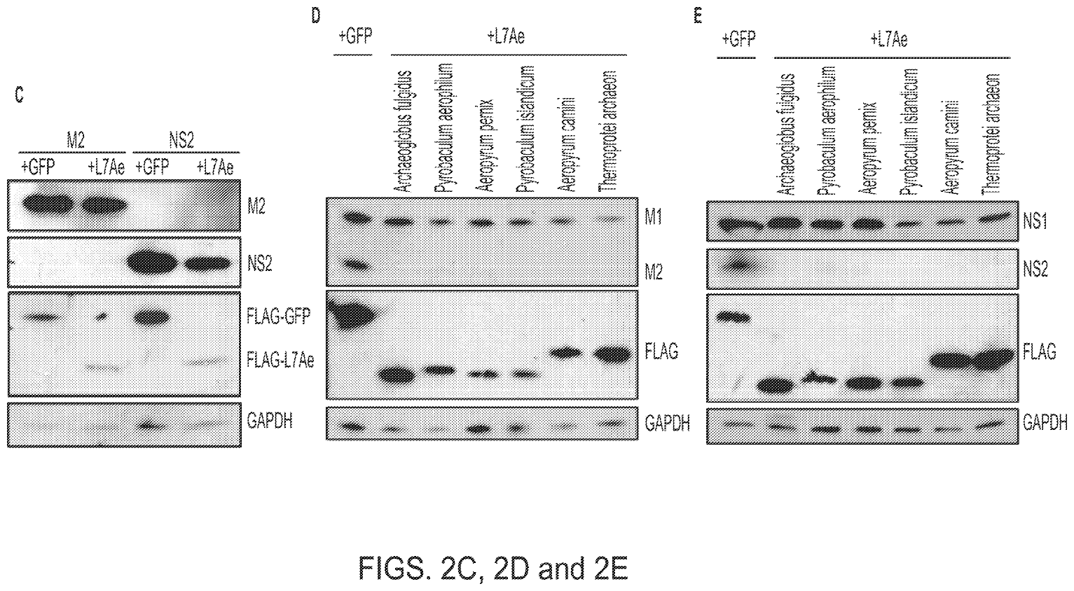

RBP11 encodes for a member of the L30 ribosomal proteins and is commonly referred to as L7Ae in archaea (Koonin, E. V., et al. Nucleic Acids Res 22, 2166-2167 (1994)). Orthologues of L7Ae can be found in all prokaryotic and eukaryotic life (Huang, L. & Lilley, D. M. RNA 19, 1703-1710 (2013)). These proteins engage so-called kink-turns (k-turns) in duplex RNA and stabilize the RNA in this secondary structure. The family of proteins includes the ribosomal proteins YbxF in bacteria, L7Ae and L30 in archaea, snu31p in yeast, and the 15.5-kDa protein of vertebrates (Watkins, N. J. et al. Cell 103, 457-466 (2000)). This family has been shown to play essential roles in site-specific modification of RNA, basic ribosomal function, and spliceosome assembly. Given the loss of splicing activity on segment eight, whether this inhibition could be observed independently of infection was determined. To this end, the three polymerase components (PA, PB1, and PB2), the nucleoprotein (NP), and viral RNA (vRNA) for segment 6 (encoding neuraminidase, NA), segment 7 (M1/M2), or segment 8 (NS1/NS2) were exogenously expressed (FIG. 1B and FIG. 2A). In the absence of L7Ae, the RNA-dependent RNA polymerase (RdRp) of the virus transcribes the NP-associated vRNA and generated robust production of the corresponding transcripts. In contrast, the addition of L7Ae from A. fulgidus completely blocked the production of M2 and NS2, while levels of NA remained unaffected. In addition, loss of M2 and NS2 in the presence of L30 also resulted in an increase in M1 and NS1 levels.

Next, to ascertain whether this inhibition required the viral RdRp or NP-associated vRNA, this study utilized DNA-dependent RNA polymerase II (Pol-II)-based plasmids expressing either NP, M1, and M2, or NS1 and NS2, controlled by the same non-canonical splice sites utilized by IAV but independent of the viral RdRp or NP. Plasmid-based expression constructs encoding NP, M1/M2 or NS1/NS2 in the presence of L7Ae demonstrated a specific inhibition of M2 and NS2 (FIG. 2B). Lastly, to verify that archaea L30 was inhibiting the processing of M2 and NS2 RNA and not protein stability, either GFP or L7Ae with non-spliced versions of M2 or NS1 was co-expressed (FIG. 2C). These data showed that in contrast to the production of these transcripts, as a result of alternative splicing, L30 had little impact on the expression of M2 and NS2 when expressed in a splicing-independent manner. Taken together, these data indicate that L7Ae interferes with the capacity of IAV to process its alternatively spliced transcripts.

Given the capacity of L7Ae to inhibit the production of M2 and NS2, a diverse set of members from distinct evolutionary branches were next cloned to ascertain whether other orthologues could phenocopy this phenomenon (Table 2). Co-expression of L30 members from bacteria, parasites, archaea, plants, arthropods, or vertebrates with segment seven or eight demonstrated that only L30 members of archaea retained the capacity to inhibit the accumulation of NS2 and M2, although some inhibitor activity from firmicutes could also be observed in the production of NS2 (FIG. 1C). To expand on this observation further, an additional five members of the L30 family from archaea were cloned. It was found that each of them could selectively inhibit the production of alternatively spliced products in the context of IAV infection (FIGS. 2D and 2E; Table 3).

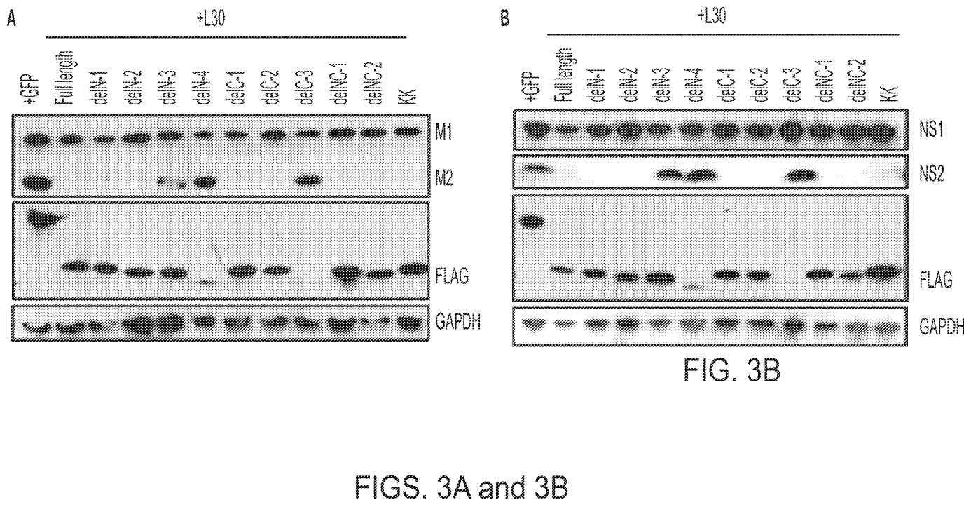

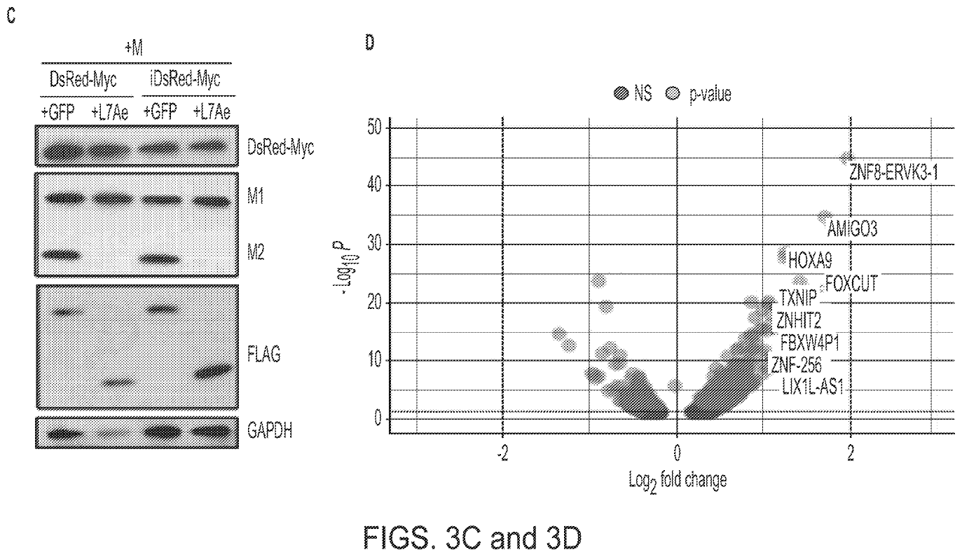

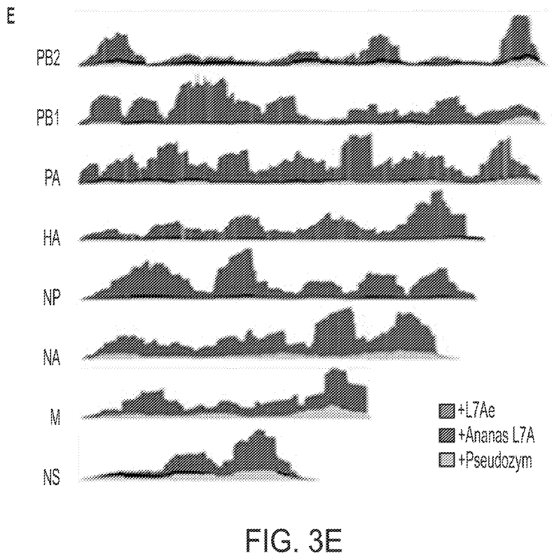

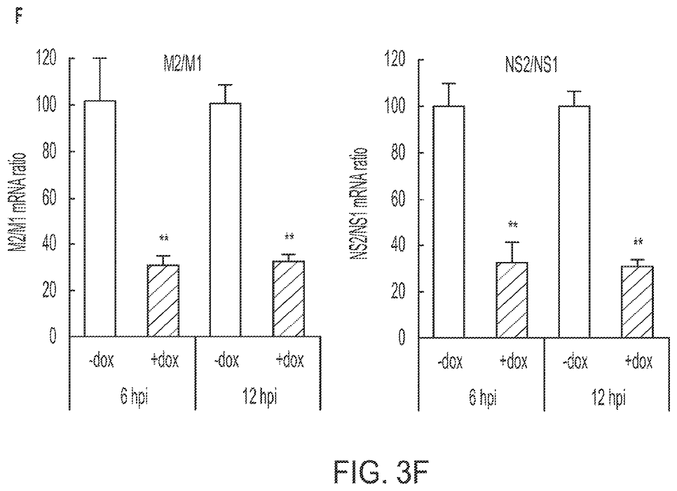

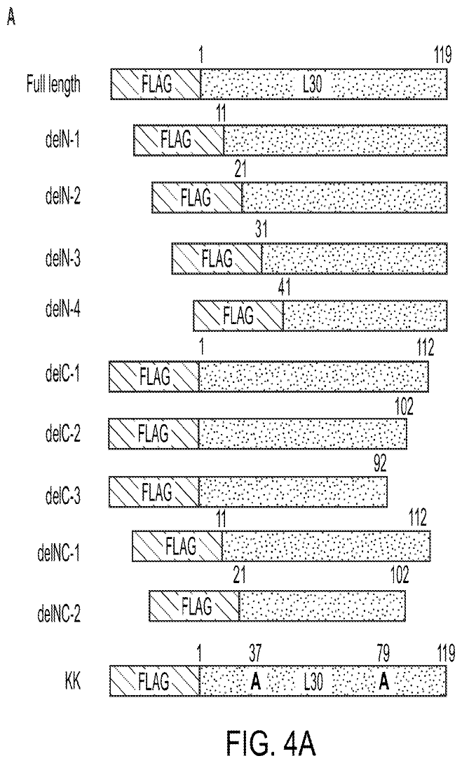

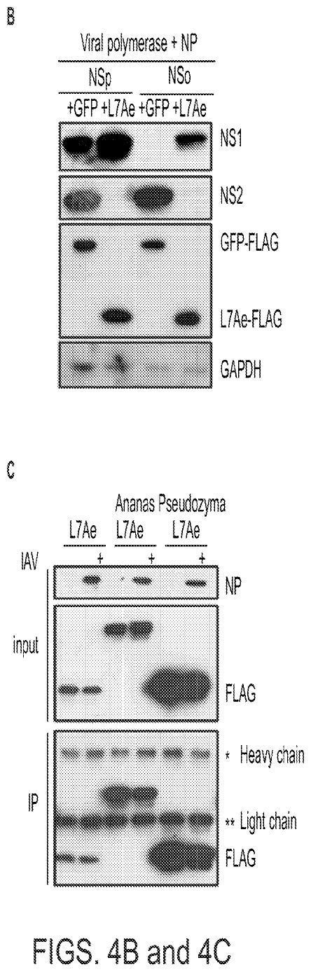

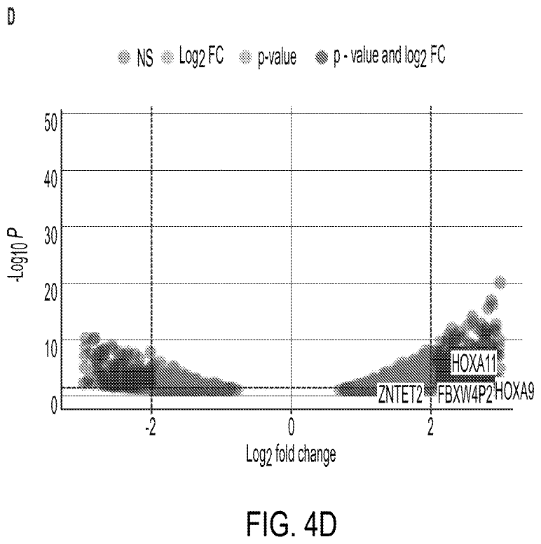

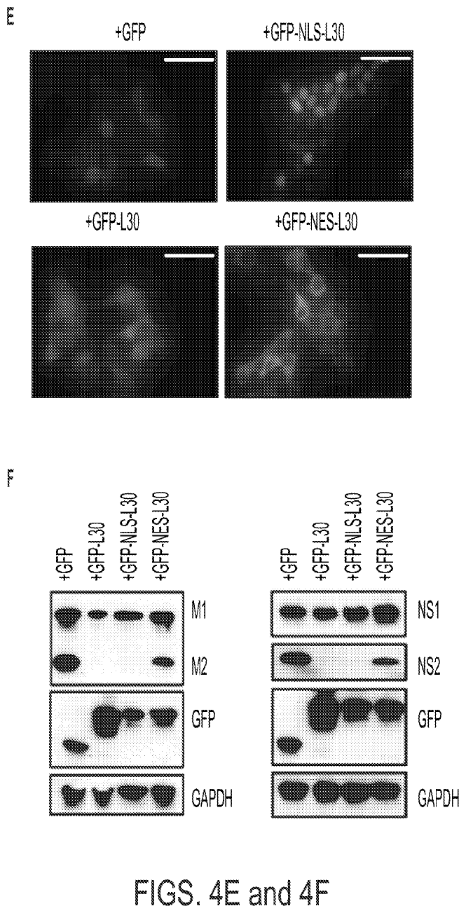

Next, to define the specific activity of L30 members from archaea, Flag-epitope tagged N- and C-terminal deletions of L7Ae from A. fulgidus were generated (FIGS. 3A and 4A). Deletion analysis of L30 demonstrated that the N-terminal 21 amino acids were dispensable for this activity as well as the C-terminal 27 residues. The remaining amino acids (21-102) represent the shared sequence of the L30 family and show near-perfect conservation across the domain of archaea. In addition, two key residues (K37 and K79) were mutated that were previously implicated in the direct engagement between L30 and k-turn RNA (FIGS. 2A, 3A, and 3B) (Saito, H. et al. Nat Chem Biol 6, 71-78 (2010)). These data demonstrate that the mutant L30 (K37A/K79A) maintained the capacity to inhibit both M2 and NS2 production, indicating that the mode of action was distinct from canonical k-turn engagement.

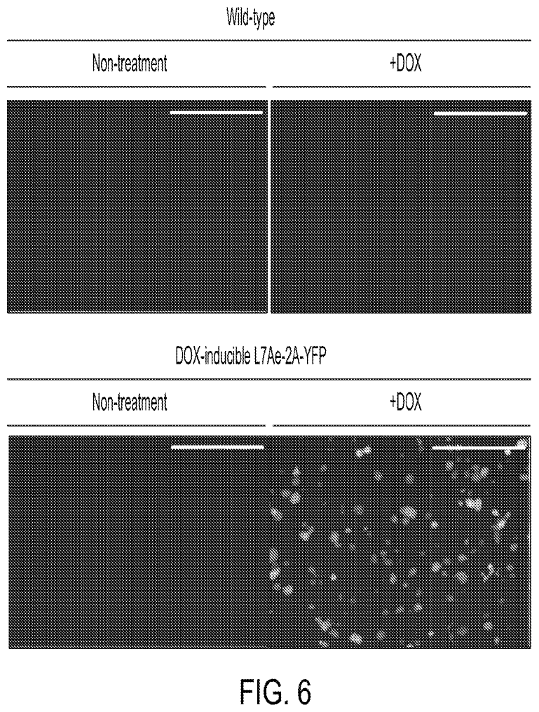

To determine the specificity for L30-mediated inhibition of M2 and NS2, this RNA binding protein was expressed in the presence of a Discosoma sp. red fluorescence gene, as either a single open reading frame (DsRed) or one containing an intron (iDsRed). In contrast to the loss of alternative splicing required to generate M2 and NS2, expression of L30 had no impact on the levels of DsRed or iDsRed (FIG. 3C). Given these results, whether L7Ae-mediated repression related to the fact that the splice sites for the generation of M2 and NS2 are sub-optimal for the host machinery was next determined (Chua, M. A., et al. Cell Rep 3, 23-29 (2013)). To this end, whether L7Ae could block the production of NS2 when the 5′ splice site was modified from its parental sequence (NSp) to the optimal (NSo) sequence AG|GURAGU was tested (Crick, F. Science 204, 264-271 (1979)). Consistent with published findings, NSp generated both NS1 and NS2, whereas NSo generated only the NS2 splice product (FIG. 6B). However, despite the canonical 5′ site utilized in the NSo vRNA, expression of L7Ae still resulted in a block of NS2 production and a corresponding increase in NS1 (FIG. 6B). Taken together, these data indicate that L7Ae engages segment 8 RNA to prevent the processing of NS2 in a manner independent of its 5′ splice site.

To ensure that L7Ae was not interfering with a subset of splicing events that included the strategy utilized by IAV to generate M2 and NS2, bulk RNA sequencing on fibroblasts was performed in the presence or absence of L7Ae. These data demonstrated that the expression of L7Ae in the absence of infection had a minimal impact on host transcripts showing the greatest differential expression on a small subset of seemingly unrelated genes (FIG. 3D and Table 2). More specifically, L7Ae expression results in an increase in reads containing a C/D box motif characterized by two terminal conserved sequences, box C (AUGAUGA) and box D (CUGA) (Ellis, J. C., et al. RNA 16, 664-666 (2010)).

Next, to determine whether this was indeed the result of direct RNA engagement, RNA immunoprecipitation-based bulk sequencing (RIP-Seq) was performed on virus-infected cells. To elucidate meaningful interactions, RIP-Seq using L30 members from archaea, ananas (pineapple), and pseudozyma (candida) was performed in IAV infected cells (FIG. 4C). These data showed that archaea L7Ae associated extensively with the vRNA of IAV in addition to a small subset of host genes that showed extensive overlap with those identified as being upregulated in the bulk RNA-Seq dataset (FIG. 3E, Table 3, and FIG. 4D). In contrast to the C/D box-containing enrichment signature, the RNA-Seq data demonstrated no obvious disruption in host transcript expression, indicating there was no overt block in splicing function. Given this data, the exon junction reads of M2 and NS2 in the presence of L7Ae were examined to ascertain whether splicing was disrupted or whether the block in protein production was the result of extensive association with the viral genome (FIG. 3D). To this end, the splice junction sites of M1, M2, NS1, and NS2 at 6 and 12 hours post infection were compared. These data found that in the presence of L7Ae, the ratio of M1:M2 or NS1:NS2 was significantly diminished to 25% of that observed for the control (FIG. 3F). Finally, to ensure L7Ae was inhibiting splicing, GFP-L7Ae constructs containing either a nuclear localization or export sequence (NLS and NES, respectively) were generated. Transfection of these constructs demonstrated the expected cellular localization patterns and further demonstrated that only L7Ae containing an NLS was capable of inhibiting IAV splicing (FIG. 4E).

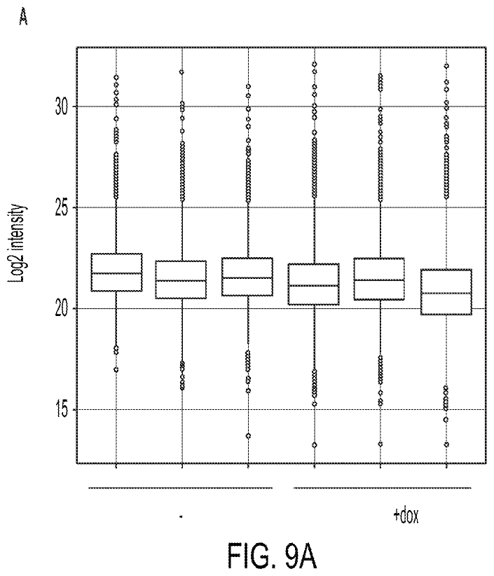

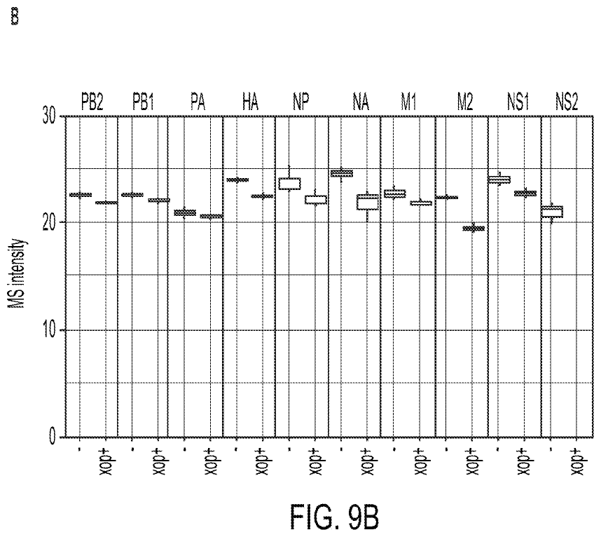

To complement RNA-Seq data that showed L7Ae does not impact host splicing, mass spectrometry (GC-TOFMS) was performed on IAV-infected human lung epithelial cells which were engineered to express L7Ae in response to doxycycline (+dox). Consistent with the RNA-Seq results, mass spectrometry-based analyses of biological triplicate samples (comparing no dox quot; −quot; to quot; +doxquot;) demonstrated no overt changes in the proteome in the presence of L7Ae with less than 1% of proteins showing any movement following doxycycline (dox) induction (FIG. 9A). In contrast, this same analysis found L7Ae expression resulted in a complete loss of NS2, a 10% loss of M2, and a ˜5% decrease in all other IAV products (FIG. 9B). Thus, L7Ae expression does not alter the host proteome but reduces IAV M2 and NS2 expression.

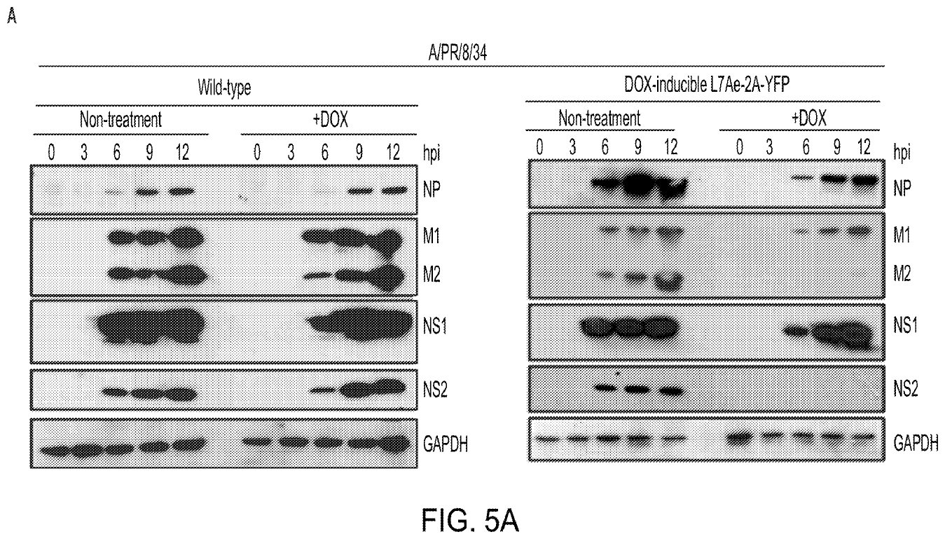

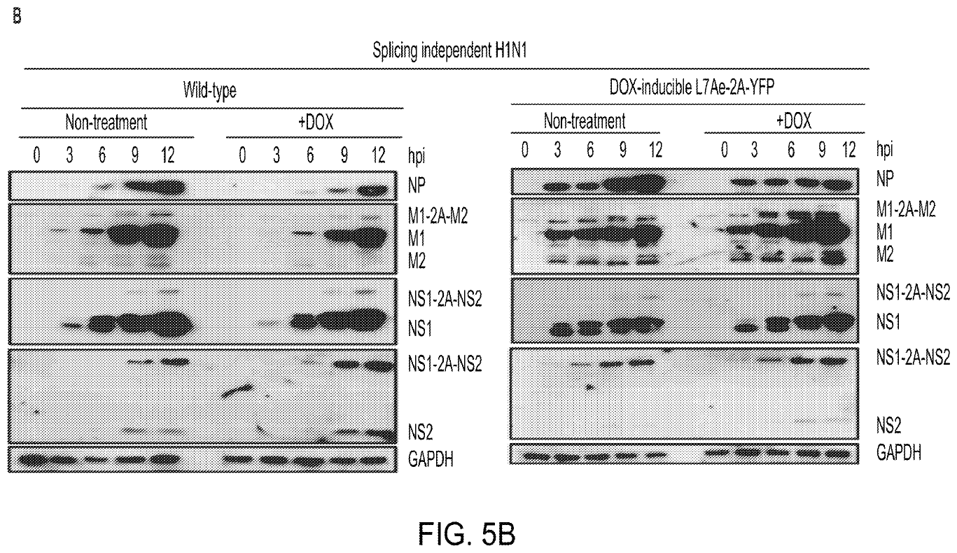

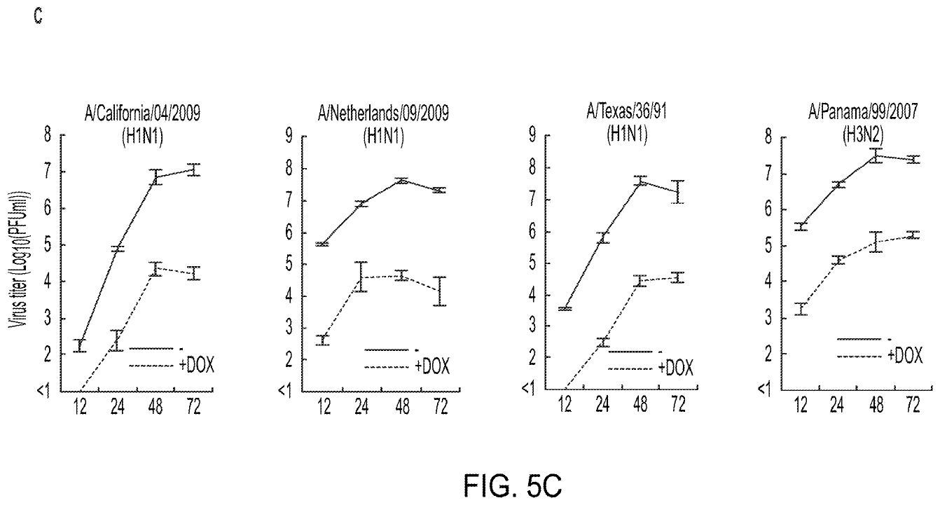

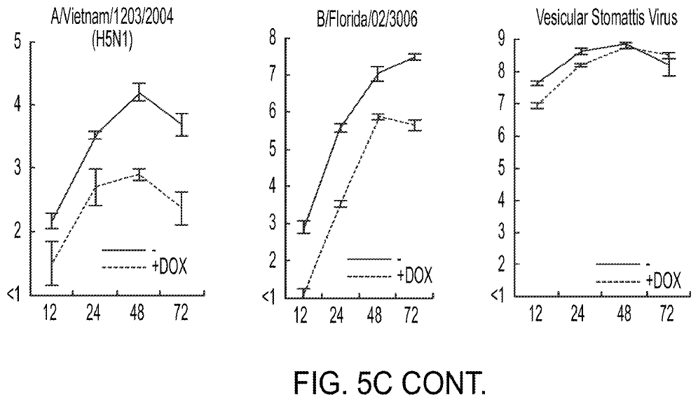

To further explore the capacity of archaea L30 proteins to inhibit IAV-based splicing, an inducible cell model system was generated. To this end, Madin-Darby canine kidney (MDCK) cells were transduced with a doxycycline (dox)-inducible system controlling the expression of L7Ae and a Yellow Fluorescent Protein (YFP) using the 2A peptide from porcine teschovirus (PTV) (Sharma, P. et al. Nucleic Acids Res 40, 3143-3151 (2012)). Following puromycin selection, YFP expression could be uniformly observed following the introduction of doxycycline (dox), which also served as a proxy for L7Ae expression that was not epitope-tagged (FIG. 6). Using unmodified MDCK cells or uninduced MDCK-L7Ae-2A-YFP cells, the addition of H1N1 A/PR/8/34 resulted in the rapid accumulation of NP, M1, M2, NS1, and NS2 as early as 6 hours post infection (hpi) (FIG. 5A). In contrast, infected MDCK-L7Ae-2A-YFP cells in the presence of dox resulted in a specific loss of the splice products M2 and NS2 (FIG. 5B). To ascertain whether L7Ae-mediated inhibition of M2 and NS2 expression could be bypassed in the absence of alternative splicing, a modified A/PR/8/34 strain was tested, in which both M2 and NS2 were generated using the same 2A element as previously described (FIG. 5C) (Nogales, A., et al. J Virol 90, 6291-6302 (2016)). In contrast to wild type virus, the splicing-independent virus demonstrated comparable levels of NP, M1, M2, NS1, and NS2 regardless of L7Ae expression. These data indicate that expression of archaeal L7Ae was disrupting some unique aspects of the biology required to generate M2 and NS2.

In an effort to discern whether this phenomenon was specific to A/Puerto Rico/8/34, H1N1 A/California/04/2009, H1N1 A/Texas/36/1991, H3N2 A/Panama/99/2007, H5N1 A/Viet Nam/1203/2004, B/Yamagata/18/1988, and an unrelated negative-strand RNA virus, Vesicular Stomatitis Virus (Indiana Strain) were additionally tested (FIG. 5C). In each influenza virus infection, expression of L7Ae successfully reduced virus titers ranging from 2-3 logs with the exception of influenza B virus infection where inhibition was significant but ranged from 1-2 logs. In contrast, L7Ae expression had no impact on titers of vesicular stomatitis virus infection.

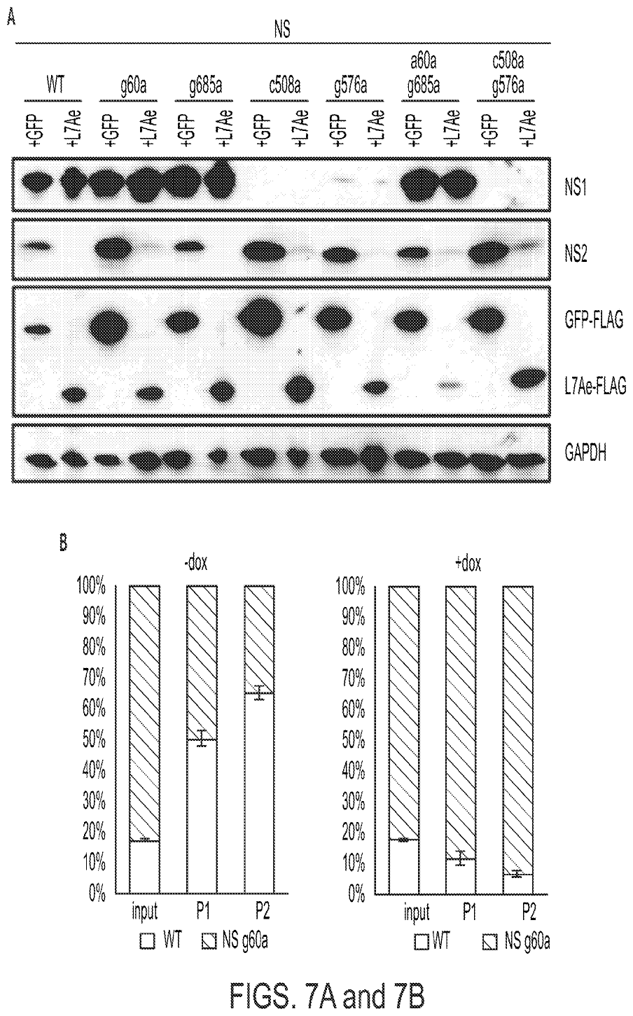

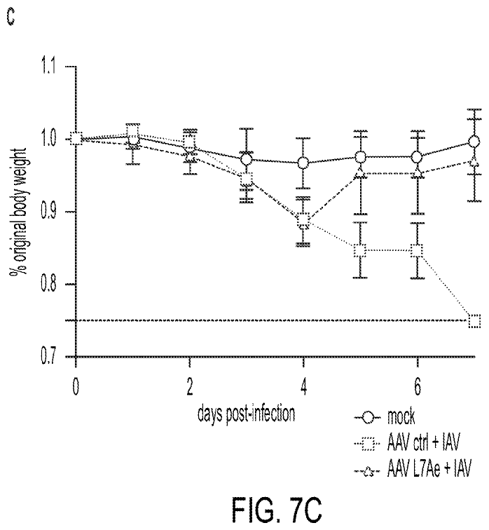

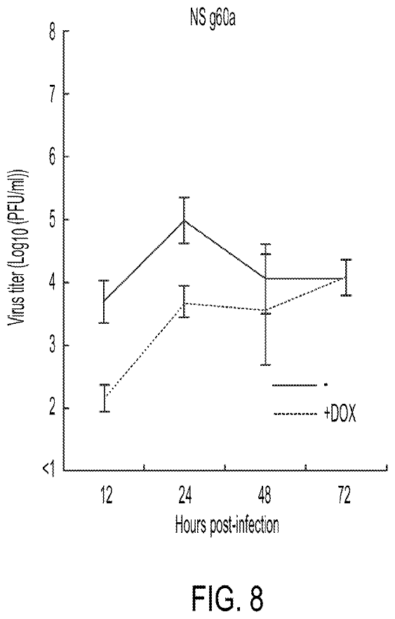

Given the broad-spectrum antiviral activity of the L30 proteins, an H1N1 virus was passaged to ascertain if viral escape mutants arose. These efforts demonstrated no observable cytopathic effect, so the virus population at passage 20 (P20) was assessed. Plaque purification of the variants present in the P20 population identified four nucleotide changes, all mapping to segment eight, including G60A, C508A, G576A, and G685A. To assess how these nucleotide polymorphisms behave in the presence of L7Ae, they were cloned as individual or combinatorial mutants into a Pol-II-based segment eight plasmid and expressed in the presence of Flag epitope-tagged GFP or L7Ae (FIG. 7A). These data demonstrated that none of the isolated mutants become fully resistant to the inhibitory activity of L7Ae, as determined by NS2 expression. Moreover, C508A and G576A mutations resulted in premature termination of NS1, a gene essential for inhibition of the host antiviral response (Ayllon, J. & Garcia-Sastre, A. Curr Top Microbiol Immunol 386, 73-107 (2015)). However, despite the inability to escape L7Ae targeting, the NS variant harboring the G60A mutant did enable a low level of NS2. However, performing growth curves on an H1N1 variant encoding this polymorphism (herein referred to as IAV_G60A), demonstrated that regardless of the presence of L7Ae, it did not reach titers that ever exceeded 10\hat{ }5 plaque forming units per ml (FIG. 8). These data were further corroborated upon assessing the virus' relative fitness. Wild type H1N1 and the IAV_G60A variant were used to coinfect MDCK cells in the absence or presence of L7Ae (FIG. 7B). Input virus populations were composed of ˜90% IAV_G60A to ˜10% wild type H1N1 and passaged to ascertain the resulting representation. These data demonstrated that in the absence of L7Ae (−dox), wild type virus rapidly outcompeted IAV_G60A by P2, whereas the mutant remained dominant in the presence of L7Ae (+dox) (FIG. 7B). Taken together, these data indicate that G60A enables enough NS2 in the presence of L7Ae to be sustained in the population, albeit with a significant loss of fitness.

Given the broad-spectrum antiviral activity of the L30 proteins, the inability of the virus to escape this restriction while maintaining fitness indicates that it represents a bona fide universal therapeutic for the family of influenza viruses provided it can be delivered to the site of infection. Of the many viral gene transfer vectors currently available, a good candidate for the genetic treatment of airway diseases is that based on the adeno-associated virus (AAV). AAV is a single-stranded virus that belongs to the Parvoviridae family and is characterized by its safety, low toxicity, and ability to confer stable expression (Carter, B. J. Mol Ther 10, 981-989 (2004)). Furthermore, of the many AAV serotypes, AAV9 has been found to efficiently transduce non-dividing alveolar epithelial cells of the lung without eliciting a humoral response, allowing for efficient re-administration of the vector (Limberis, M. P. & Wilson, J. M. Proc Natl Acad Sci USA 103, 12993-12998 (2006)). To this end, either YFP or L7Ae-2A-YFP was introduced into AAV9 and administered it one week prior to a lethal challenge of A/Puerto Rico/8/1934 (FIG. 7C). This experimental model found that AAV administration was well tolerated and yielded no weight loss in the mice. Upon IAV challenge, AAV9 YFP-treated mice demonstrated dramatic morbidity with extensive weight loss over a seven-day period that ultimately resulted in 100% mortality. In contrast, the cohort treated with AAV9 expressing L7Ae-2A-YFP showed only modest weight loss on day 4 followed by complete recovery (FIG. 7C). These data corroborate the findings that L7Ae can effectively inhibit IAV biology and could be adapted as a universal therapeutic.

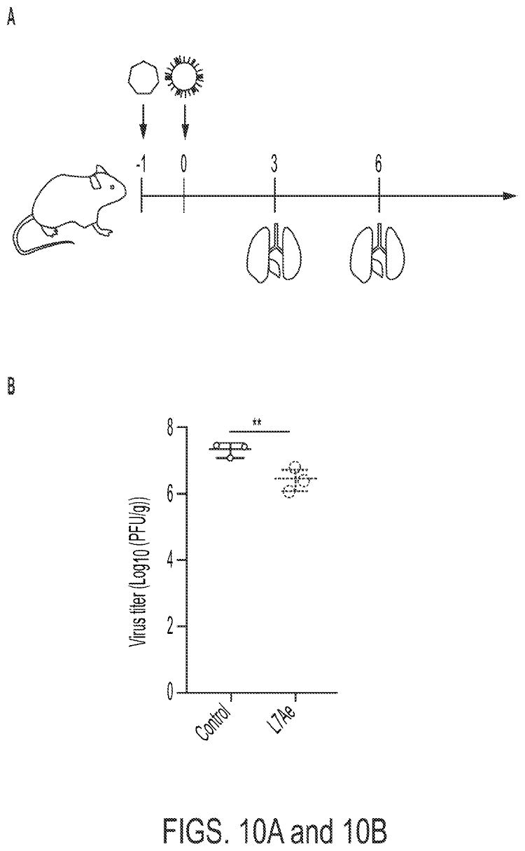

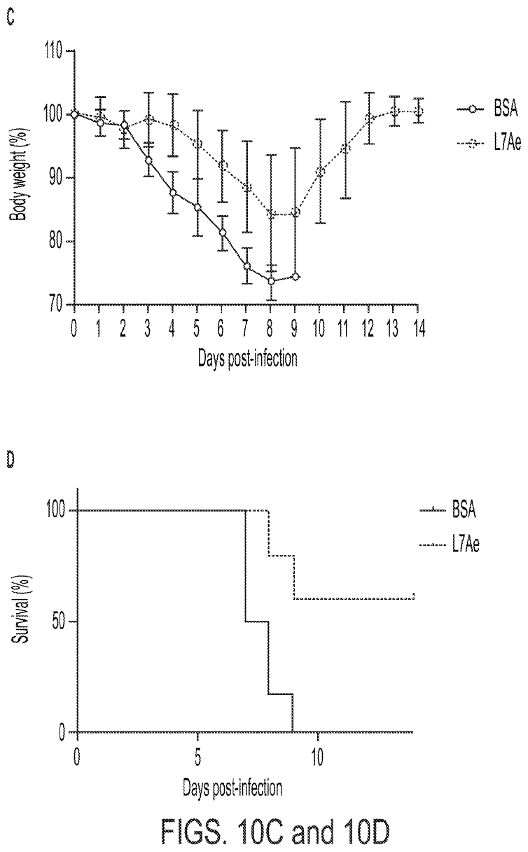

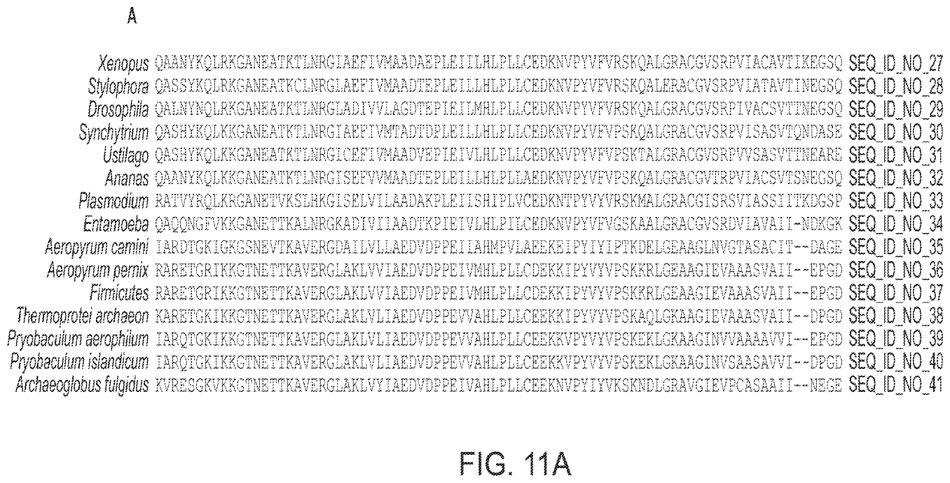

To further demonstrate in vivo efficacy, recombinant full-length L7Ae or bovine serum albumin (BSA) were administered to BL/6 female mice, 4-6 weeks of age. BSA and L7Ae were mixed with the lipid-based protein transfection reagent (Pierce Transfection Reagent) as per the instructions of ThermoScientific. Protein:lipid mixtures were administered as a single intranasal dose of ten micrograms one day prior to challenge with a mouse-adapted H1N1 stain (A/Puerto Rico/08/1934). Mice were infected with 250 plaque forming units, a dose corresponding to five times the 50% lethal dose (5MLD50). Cohorts of six animals per condition were monitored for weight loss daily (as a proxy for morbidity), and an additional three animals were treated per cohort and sacrificed on day three to determine viral load (FIG. 10A). These data demonstrate that on day three, L7Ae-treated animals showed a ˜1 log reduction in viral titer (FIG. 10B). Longitudinal studies on the remaining six animals per cohort demonstrated all six BSA-treated animals reached their ethical endpoints between days 7-9 and required euthanization. In contrast, L7Ae-treated animals showed minimal signs of weight loss overall, reaching full recovery weights by the end of the experiment (FIG. 10C). Measuring mortality across cohorts also demonstrates that a single dose of L7Ae significantly reduces mortality (FIG. 10D).

### DISCUSSION

The discovery that members of the archaea L30 family can specifically block splicing regardless of strain provides a new modality for the development of such a universal therapeutic. Here, it was demonstrated that despite the fact that all life encodes an orthologue of L30, only members of the archaea domain have been found to interfere specifically with IAV splicing. Based on RIP-Seq efforts and attempts to generate an escape mutant, the findings support the hypothesis that Archaea L30 protein engages the 5′ splice sites of segment 7 and 8 and prevents the transesterification of the 5′ donor site. As inhibition of host splicing events was not observed, the data further indicate that the RNA of segment 7 and 8 must adapt a distinct form that enables L30 engagement. As RIP-Seq data indicated that archean L30 shows a high affinity for various aspects of the viral vRNA, it was postulated that the viral ribonucleoprotein structure might prove to be a substrate for this family of proteins and that engagement on segment 7 and 8 specifically inhibits M2 and NS2. This concept is also supported by the fact that the G60A mutant that arose during passaging in the presence of L30E did not become resistant to L30, but rather the mutation made splicing more efficient to ensure enough NS2 was generated. These data are in line with earlier findings that have demonstrated that NS2 serves as an integral timer that controls the later aspects of IAV biology (Chua, M. A., et al. Cell Rep 3, 23-29 (2013)).

The capacity to block the production of both MS2 and NS2 would have profound impacts on virus biology. Amantadines, a small molecule M2 blocker from which IAV rapidly escapes, has been found to prevent viral uncoating upon entry, and thus the RNP fails to launch and promote infection. Given that the P20 escape mutant did not show mutants in segment 7, undetectable levels of M2 may be sufficient to enable virus entry. In contrast to M2, loss of NS2 results in a delay in the switch to transcription to replication as well the export of the viral RNA segment (Chua, M. A., et al. Cell Rep 3, 23-29 (2013)). The G60A mutation thus compensated for L30-mediated inhibition of splicing by adapting a canonical splice site, but this adaptation comes at a fitness cost as it also results in low levels of NS1 and inappropriate high concentrations of NEP in non-L30-expressing cells. Together, these dynamics indicate that a bona fide escape from L30 by a mutant virus that maintains its fitness is unlikely.

Delivery of L30 likely serves both as a prophylactic or a therapeutic for all strains of the orthomyxovirus family. As expression of L30e does not impact host cell biology, the application of an AAV vector could protect the host for months, possibly years depending on the persistence of the vector. Alternatively, other temporal delivery platforms, including liposomes, cell-penetrating peptides, and/or virus-like particles, may also be used to safely deliver L7Ae to the upper respiratory tract at the time of infection to reduce both morbidity and mortality rates. In all, the data demonstrated that this 80-amino acid protein is highly efficacious against otherwise lethal influenza virus infections both in vitro and in vivo.

TABLE 1
The list of the RNA-binding proteins that used in the screening

| | Protein | Organism | Accession |
|---|---|---|---|
| RBP1 | RNA-binding protein | Candidatus Pacearchaeota | PIN90574 |
| | | archaeon | |
| RBP2 | RNA-binding protein | Methanomicrobiales archaeon | PKL59568 |
| | | HGW-Methanomicrobiales-2 | |
| RBP3 | 50S ribosomal protein L23 | Nanoarchaeum equitans | AAR38920 |
| RBP4 | RNP-1 like RNA-binding | Methanoplanus limicola | WP_004076397 |
| | protein | | |
| RBP5 | RNA-binding protein | Euryarchaeota archaeon | PBO81357 |
| RBP6 | RNA-binding protein | Methanomicrobiales archaeon | PKL61235 |
| RBP7 | RNA recognition motif- | Methanoculleus bourgensis | WP_014868175 |
| | containing protein | | |
| RBP8 | RNP-1 like RNA-binding | Methanofollis liminatans | EJG06207 |
| | protein | | |
| RBP9 | RNP-1 like RNA-binding | Methanofollis liminatans | WP_004039180 |
| | protein | | |
| RBP10 | RNA-binding region RNP-1 | Methanospirillum hungatei | WP_004039180 |
| RBP11 | 50S ribosomal protein L7Ae | Archaeoglobus fulgidus | WP_01087826 |
| RBP12 | Ribonuclease III | Candidatus | KYC45091 |
| | | Methanofastidiosum | |
| | | methylthiophilus | |
| RBP13 | RNA-binding region RNP-1 | Methanospirillum hungatei | ABD41297 |
| | | JF-1 | |
| RBP14 | Ribonuclease 3 | Methanobacterium paludis | WP_013825713 |
| RBP15 | Ribonuclease 3 | Methanobacterium | CDG65126 |
| RBP16 | Ribonuclease III Rnc | Methanobrevibacter | WP_012956077 |
| | | ruminantium | |
| RBP17 | DsRNA-specific ribonuclease | Methanolobus tindarius | WP_023846615 |
| RBP18 | 3′-to-5′ exoribonuclease | Methanosarcina sp. | WP_048129205 |
| | RNase R | | |
| RBP19 | Exoribonuclease II | Methanosarcina sp. | WP_048169923 |
| RBP20 | Exoribonuclease II | Methanosarcina mazei | AAM30240 |
| RBP21 | 3′-to-5′ exoribonuclease | Methanosarcina | AKB77354 |
| | RNase R | horonobensis | |

TABLE 2
L30 members from different organisms

| | | | % aa sequence identity |
|---|---|---|---|
| | Organism | Accession | to SEQ ID NO: 1 |
| Plasmodium | Plasmodium gonderi | XP_028543596 | 41% |
| Entamoeba | Entamoeba dispar SAW760 | XP_001739790 | 49% |
| Firmicutes | Firmicutes bacterium | OPZ88532 | 53% |
| | ADurb.Bin419 | | |
| Archaeoglobus | Archaeoglobus fulgidus | WP_010878267 | 100% |
| Pyrobaculum | Pyrobaculum sp. OCT 11 | WP_012350923 | 64% |
| Synchytrium | Synchytrium microbalum | XP_031023043 | 41% |
| Ustilago | Ustilago maydis 521 | XP_011392655 | 45% |
| Ananas | Ananas comosus | XP_020103891 | 42% |
| Stylophora | Stylophora pistillata | XP_022780273 | 47% |
| Xenopus | Xenopus tropicalis | NP_988994 | 45% |

TABLE 3
L30 members from archaea

| Organism | Accession |
|---|---|
| Archaeoglobus fulgidus | WP_010878267 |
| Pyrobaculum aerophilum | WP_116420300 |
| Aeropyrum pernix | WP_010866613 |
| Pyrobaculum islandicum | WP_053240293 |
| Aeropyrum camini | WP_022541879 |
| Thermoprotei archaeon | RLE78096 |

TABLE 4
Differentially expressed genes induced by L30

| Gene Name | baseMean | log2FoldChange | lfcSE | stat | pvalue | padj |
|---|---|---|---|---|---|---|
| ZNF8-ERVK3-1 | 231.608082 | 1.965979151 | 0.128058088 | 14.79402094 | 1.60E−49 | 2.52E−45 |
| AMIGO3 | 392.5790465 | 1.70875086 | 0.128786344 | 13.09895213 | 3.34E−39 | 2.63E−35 |
| FOXCUT | 226.3059284 | 1.418538395 | 0.128250412 | 10.88770978 | 1.32E−27 | 3.47E−24 |
| FBXW4P1 | 74.02704474 | 1.288106436 | 0.145892632 | 8.494125744 | 1.99E−17 | 1.43E−14 |
| HOXA9 | 1713.747494 | 1.253667207 | 0.104694642 | 11.95699475 | 5.97E−33 | 3.14E−29 |
| HOXA11 | 749.8600608 | 1.240514254 | 0.104984856 | 11.77727222 | 5.11E−32 | 2.01E−28 |
| LIX1L-AS1 | 38.42130941 | 1.227647536 | 0.144275281 | 7.193327166 | 6.32E−13 | 2.37E−10 |
| DNAJB4 | 287.0038113 | −0.500760424 | 0.12626752 | −3.961784211 | 7.44E−05 | 0.002910139 |
| FAM72C | 1249.295322 | −0.503411929 | 0.095270847 | −5.285278259 | 1.26E−07 | 1.28E−05 |
| KIFC1 | 1423.712025 | −0.506461924 | 0.078111703 | −6.484698904 | 8.89E−11 | 1.96E−08 |
| FBXO48 | 57.12287151 | −0.509784653 | 0.149831662 | −3.406533126 | 0.000657936 | 0.015098043 |
| UBE2W | 485.7312916 | −0.511641957 | 0.109552986 | −4.671426639 | 2.99E−06 | 0.00019934 |
| CCDC84 | 198.5059165 | −0.518780475 | 0.127579485 | −4.072342492 | 4.65E−05 | 0.001946274 |
| N4BP2L1 | 44.51236981 | −0.521628371 | 0.149512589 | −3.489893056 | 0.000483214 | 0.012111076 |
| TMEM140 | 219.0801011 | −0.524986017 | 0.114666945 | −4.577983929 | 4.69E−06 | 0.000289115 |
| CDCA2 | 631.7183253 | −0.527102548 | 0.086524652 | −6.092886013 | 1.11E−09 | 1.96E−07 |
| ZNF738 | 429.1134688 | −0.529119404 | 0.122979465 | −4.311298871 | 1.62E−05 | 0.000830725 |
| LOC100128288 | 21.64720188 | −0.533209638 | 0.141752723 | −3.768320426 | 0.00016435 | 0.005420444 |
| MYLK3 | 27.9448519 | −0.534875664 | 0.140302281 | −3.803362978 | 0.000142745 | 0.00486042 |
| MCEE | 105.9042599 | −0.535585767 | 0.137401416 | −3.898984071 | 9.66E−05 | 0.003591636 |
| GYS2 | 12.79156657 | −0.535636231 | 0.118381604 | −4.730269738 | 2.24E−06 | 0.000155721 |
| CALHM1 | 9.66783117 | −0.535718892 | 0.114575039 | −4.567312709 | 4.94E−06 | 0.000299545 |
| MYLK2 | 23.89042891 | −0.540294898 | 0.145321597 | −3.709955681 | 0.000207296 | 0.006333361 |
| C1orf216 | 200.932218 | −0.560150952 | 0.122834104 | −4.56439681 | 5.01E−06 | 0.00030142 |
| ZNF8 | 122.0422589 | −0.561335785 | 0.148564992 | −3.772054386 | 0.000161909 | 0.005373673 |
| AATBC | 70.75492054 | −0.563541921 | 0.14963453 | −3.76216422 | 0.000168449 | 0.00551336 |
| GATD3A | 1540.909504 | −0.575129207 | 0.114031889 | −5.044151607 | 4.56E−07 | 3.92E−05 |
| ERN2 | 15.00246472 | −0.580538869 | 0.122807177 | −4.910940164 | 9.06E−07 | 7.11E−05 |
| MLH3 | 84.19988403 | −0.581639499 | 0.141911775 | −4.093780391 | 4.24E−05 | 0.001817842 |
| TMEM161B-AS1 | 57.67976271 | −0.583001689 | 0.148894345 | −3.927304992 | 8.59E−05 | 0.003247629 |
| SEC31B | 313.7162374 | −0.5831651 | 0.113099775 | −5.154244655 | 2.55E−07 | 2.33E−05 |
| GNB3 | 109.1578083 | −0.584835437 | 0.134722174 | −4.341476689 | 1.42E−05 | 0.000736368 |
| FOXD4L1 | 14.07274045 | −0.625060814 | 0.128630968 | −4.852268415 | 1.22E−06 | 9.21E−05 |
| H3F3A | 189.2296065 | −0.630000361 | 0.147761907 | −4.279663449 | 1.87E−05 | 0.000925026 |
| FOXD4L6 | 21.95982788 | −0.647005895 | 0.125381407 | −5.574616826 | 2.48E−08 | 3.01E−06 |
| HNRNPH2 | 1297.096929 | −0.654653169 | 0.086412764 | −7.575478895 | 3.58E−14 | 1.57E−11 |
| UBE2C | 2540.887679 | −0.655811388 | 0.091970489 | −7.132002936 | 9.89E−13 | 3.54E−10 |
| CYP2T1P | 37.54325784 | −0.656521626 | 0.149861448 | −4.365106337 | 1.27E−05 | 0.00067445 |
| DLEU2 | 208.410974 | −0.693346742 | 0.123968801 | −5.599915495 | 2.14E−08 | 2.68E−06 |
| CKS2 | 2070.239988 | −0.701234374 | 0.098987883 | −7.086079796 | 1.38E−12 | 4.35E−10 |
| FOXD4L4 | 13.6929412 | −0.730420975 | 0.124931928 | −5.26017532 | 1.44E−07 | 1.45E−05 |
| SNHG4 | 488.7504616 | −0.765350472 | 0.095761838 | −7.990218905 | 1.35E−15 | 7.58E−13 |
| CNKSR1 | 49.26699679 | −0.784109049 | 0.149735288 | −5.219465679 | 1.79E−07 | 1.77E−05 |
| SNHG3 | 2876.583188 | −0.813294133 | 0.082111741 | −9.905755533 | 3.93E−23 | 6.20E−20 |
| BCL2L2-PABPN1 | 433.5321497 | −0.854266832 | 0.111120664 | −7.682227246 | 1.56E−14 | 7.25E−12 |
| CSTF3 | 1282.267008 | −0.896891759 | 0.082076309 | −10.93099487 | 8.19E−28 | 2.58E−24 |
| NME1-NME2 | 468.5813151 | −0.898477524 | 0.142836723 | −6.310944136 | 2.77E−10 | 5.47E−08 |
| TAF7L | 48.81901052 | −0.932741928 | 0.149938789 | −6.173373208 | 6.68E−10 | 1.23E−07 |
| GCNA | 63.6470952 | −0.950145229 | 0.147033764 | −6.416151147 | 1.40E−10 | 2.94E−08 |
| FOXD4 | 53.5400666 | −0.975375938 | 0.148900255 | −6.506187094 | 7.71E−11 | 1.74E−08 |
| FOXD4L5 | 35.09958363 | −1.241460434 | 0.147032551 | −8.125379861 | 4.46E−16 | 2.81E−13 |
| BAAT | 39.90438176 | −1.353549893 | 0.149467344 | −8.695198738 | 3.46E−18 | 3.03E−15 |

TABLE 5
L30 associated RNAs (L2F > 3)

| Gene Name | baseMean | log2FoldChange | lfcSE | stat | pvalue | padj |
|---|---|---|---|---|---|---|
| FAM69A | 88.41451508 | 4.982011671 | 0.560966311 | 8.532578339 | 1.43E−17 | 4.68E−15 |
| MIR4453HG | 30.04461585 | 4.624248373 | 0.733535715 | 5.100117671 | 3.39E−07 | 1.42E−05 |
| GDF9 | 35.45027277 | 4.41593468 | 0.666285225 | 5.604505445 | 2.09E−08 | 1.15E−06 |
| MGC12916 | 38.45272662 | 4.358436751 | 0.663390873 | 5.578709373 | 2.42E−08 | 1.30E−06 |
| P2RY11 | 19.18635191 | 4.340278451 | 0.815271596 | 4.960487207 | 7.03E−07 | 2.66E−05 |
| TRARG1 | 13.17970666 | 4.293908823 | 0.827232006 | 4.871569716 | 1.11E−06 | 3.90E−05 |
| LOC100129931 | 19.07456128 | 4.230177348 | 0.757421493 | 4.721450525 | 2.34E−06 | 7.63E−05 |
| SNORA73B | 3646.064166 | 4.083895989 | 0.481154475 | 8.483918747 | 2.18E−17 | 6.75E−15 |
| UTP3 | 258.1127089 | 4.082185005 | 0.433848147 | 9.308515898 | 1.30E−20 | 6.26E−18 |
| LOC102724580 | 11.16961854 | 4.070509853 | 0.837226338 | 4.614361667 | 3.94E−06 | 0.000118377 |
| LOC100507599 | 69.25395401 | 4.048082797 | 0.534662144 | 7.22407977 | 5.05E−13 | 6.92E−11 |
| HEBP2 | 186.6002572 | 4.024003221 | 0.490851923 | 8.061055741 | 7.56E−16 | 1.83E−13 |
| COMMD10 | 13.27539441 | 4.02088658 | 0.748471281 | 5.15029174 | 2.60E−07 | 1.11E−05 |
| LOC93622 | 226.9740944 | 3.95029141 | 0.392793976 | 9.934231394 | 2.95E−23 | 2.05E−20 |
| LIX1L-AS1 | 10.98752888 | 3.848125505 | 0.844313363 | 4.365610595 | 1.27E−05 | 0.000301529 |
| TSPYL4 | 140.5450426 | 3.829763468 | 0.422017534 | 8.916700302 | 4.80E−19 | 1.84E−16 |
| RNF113A | 143.1322426 | 3.6908834 | 0.476940757 | 7.623212559 | 2.47E−14 | 4.23E−12 |
| ACTG1P20 | 19.86596035 | 3.653680298 | 0.70402558 | 4.572892547 | 4.81E−06 | 0.000137814 |
| CCDC87 | 7.472366852 | 3.632404362 | 0.859625772 | 4.100884848 | 4.12E−05 | 0.000792642 |
| LOC651337 | 11.9883604 | 3.61401478 | 0.795203964 | 4.165287876 | 3.11E−05 | 0.000627045 |
| ST6GALNAC2 | 18.06466312 | 3.596620572 | 0.743429989 | 4.212901598 | 2.52E−05 | 0.000530388 |
| CAPN10-DT | 57.72704689 | 3.477029596 | 0.571982929 | 5.882336614 | 4.05E−09 | 2.54E−07 |
| PRTFDC1 | 70.93169478 | 3.450144333 | 0.520553006 | 6.480557219 | 9.14E−11 | 8.60E−09 |
| FBXW7 | 170.394364 | 3.438486857 | 0.44421803 | 7.720708465 | 1.16E−14 | 2.14E−12 |
| FOXCUT | 46.46608605 | 3.423559036 | 0.576647368 | 5.666563589 | 1.46E−08 | 8.26E−07 |
| SEC14L1P1 | 18.34922446 | 3.417083532 | 0.71457556 | 4.338692652 | 1.43E−05 | 0.000333782 |
| ZNF8-ERVK3-1 | 122.8767809 | 3.411237713 | 0.433204609 | 7.761534448 | 8.39E−15 | 1.61E−12 |
| LINC00115 | 22.71738678 | 3.350479551 | 0.693605324 | 4.418979059 | 9.92E−06 | 0.000244791 |
| CENPBD1 | 79.17902084 | 3.34757691 | 0.472754144 | 6.933342836 | 4.11E−12 | 4.81E−10 |
| GCC1 | 123.6029004 | 3.336476845 | 0.413544436 | 7.959753977 | 1.72E−15 | 3.83E−13 |
| TIGD1 | 44.28552316 | 3.332900364 | 0.567248424 | 5.677418593 | 1.37E−08 | 7.83E−07 |
| SNORA62 | 169.8190557 | 3.319485646 | 0.831087674 | 4.03711644 | 5.41E−05 | 0.000982154 |
| RECK | 12.56345423 | 3.319303447 | 0.736451985 | 4.258747972 | 2.06E−05 | 0.000446874 |
| CT45A10 | 6.296084376 | 3.309064557 | 0.873255255 | 3.725453753 | 0.000194964 | 0.002769389 |
| ZNF830 | 90.76291848 | 3.305904043 | 0.443279691 | 7.339483914 | 2.14E−13 | 3.18E−11 |
| CWC27 | 235.3458857 | 3.299383753 | 0.458274991 | 7.169247155 | 7.54E−13 | 1.01E−10 |
| CYB5D1 | 195.8008982 | 3.287120221 | 0.367205171 | 8.889688501 | 6.13E−19 | 2.27E−16 |
| RNVU1-20 | 72.15938955 | 3.283191007 | 0.64235905 | 4.992788454 | 5.95E−07 | 2.29E−05 |
| SNORA79B | 53.81535429 | 3.276850324 | 0.566604969 | 5.599668116 | 2.15E−08 | 1.18E−06 |
| BIVM-ERCC5 | 6.537963526 | 3.267745147 | 0.875753525 | 3.685129221 | 0.000228587 | 0.003154878 |
| RRS1 | 199.4427422 | 3.238824874 | 0.392285346 | 8.194309243 | 2.52E−16 | 6.51E−14 |
| NPM1 | 11119.3406 | 3.220266167 | 0.353622362 | 9.105959312 | 8.55E−20 | 3.65E−17 |
| CHMP1B | 75.05398077 | 3.218455759 | 0.46136367 | 6.850410781 | 7.36E−12 | 8.10E−10 |
| KYAT3 | 48.32355919 | 3.205744541 | 0.624692082 | 5.001030648 | 5.70E−07 | 2.21E−05 |
| MXD3 | 43.73928396 | 3.153699579 | 0.56775103 | 5.355331024 | 8.54E−08 | 4.11E−06 |
| GALNT4 | 20.89648212 | 3.134708378 | 0.727979689 | 4.027103542 | 5.65E−05 | 0.00101826 |
| LOC100128653 | 5.034379429 | 3.133625176 | 0.883872088 | 3.515243943 | 0.00043935 | 0.005387499 |
| C2orf68 | 27.71136311 | 3.103124231 | 0.603190234 | 4.892897718 | 9.94E−07 | 3.60E−05 |
| PURB | 89.78639398 | 3.102911529 | 0.443274411 | 6.906854843 | 4.96E−12 | 5.62E−10 |
| EXOC8 | 288.0561144 | 3.08759455 | 0.436423777 | 7.053966048 | 1.74E−12 | 2.08E−10 |
| CSTF2T | 546.2611366 | 3.080856886 | 0.32444008 | 9.480156981 | 2.54E−21 | 1.48E−18 |
| ASDURF | 6.339541312 | 3.055336834 | 0.879829362 | 3.428911459 | 0.000606007 | 0.006954058 |
| CETN3 | 30.17084927 | 3.020023127 | 0.705447883 | 4.196647437 | 2.71E−05 | 0.0005614 |
| ZNF548 | 35.89352143 | 3.005839482 | 0.555952932 | 5.235227296 | 1.65E−07 | 7.41E−06 |

What is claimed is:

1. A method for preventing or treating an influenza virus infection or influenza virus disease in a subject, comprising administering to a subject in need thereof a nucleic acid molecule comprising a polynucleotide encoding an L7Ae protein or a fragment/variant thereof or administering to the subject in need thereof the L7Ae protein or fragment/variant thereof.

2. The method of claim 1, wherein: (i) the L7Ae protein comprises an amino acid sequence having at least 75% identity to any one of SEQ ID NOs: 1-10, or comprises an amino acid sequence of SEQ ID NOs: 1-10; or (ii) the L7Ae protein comprises an amino acid sequence having at least 63% identity to the amino acid sequence of SEQ ID NO: 4 or SEQ ID NO: 1, or comprises the amino acid sequence of SEQ ID NO: 4 or SEQ ID NO: 1, (a) optionally wherein the L7Ae protein comprises an I or L at position 88, an E or N at position 89, or a V at position 90, or combinations thereof, wherein the position is relative to SEQ ID NO:1, (b) optionally wherein the L7Ae protein comprises an I or L at position 88, an E or N at position 89, and a V at position 90, wherein the position is relative to SEQ ID NO:1, (c) optionally wherein the L7Ae protein comprises an I at position 88, an E at position 89, and a V at position 90, (d) optionally wherein the L7Ae protein comprises an I at position 88, an N at position 89, and a V at position 90, (e) optionally wherein the L7Ae protein comprises an L at position 88, an N at position 89, and a V at position 90, (f) optionally wherein the L7Ae protein comprises an L at position 88, an E at position 89, and a V at position 90, (g) optionally wherein the L7Ae protein does not comprise a R at position 90, (h) optionally wherein the L7Ae protein further comprises one or more of amino acid residues N33, E34, K37, R41, and K79, wherein the position is relative to SEQ ID NO:1, or (i) optionally wherein the L7Ae protein comprises an I or L at position 88, an E or N at position 89, a V at position 90, an N at position 33, an E at position 34, a K at position 37, an R at position 41, and a K at position 79, wherein the position is relative to SEQ ID NO:1.

3. The method of claim 1 or 2, wherein the polynucleotide comprises a nucleotide sequence having at least 75% identity to any one of SEQ ID NOs: 11-15, or comprises a nucleotide sequence of SEQ ID NOs: 11-15.

4. The method of any one of the preceding claims, wherein the nucleic acid molecule comprises a viral vector.

5. The method of claim 4, wherein the viral vector comprises an adeno-associated viral vector, lentiviral vector or adenoviral vector.

6. The method of claim 5, wherein the adeno-associated viral vector is selected from the group consisting of AAV1, AAV2, AAV3, AAV4, AAV5, AAV6, AAV7, AAV8, AAV9, AAV10, AAV11, AAV 12, AAV13, AAV rh74, and recombinant subtypes thereof.

7. The method of any one of the preceding claims, wherein the influenza virus infection or influenza virus disease is associated with an influenza A virus, an influenza B virus, an influenza C virus, or an Isavirus.

8. The method of claim 7, wherein the influenza A virus comprises a serotype selected from the group consisting of H1N1, H2N2, H3N1, H3N2, H5N1, H7N7, H1N2, H9N2, H7N2, H7N3, H10N7, H7N9, and H6N1.

9. The method of any one of the preceding claims, wherein the subject is a mammal, a fish, or an avian.

10. The method of any one of the preceding claims, wherein the subject is human.

11. The method of any one of the preceding claims, wherein the nucleic acid molecule or the L7Ae protein is administered to the subject prior to the onset of an influenza season.

12. The method of any one of the preceding claims, comprising administrating to the subject a second therapeutic agent or therapy.

13. The method of claim 12, wherein the second therapeutic agent comprises an antiviral agent.

14. The method of claim 13, wherein the antiviral agent is selected from the group consisting of Oseltamivir, Zanamivir, Amantadine, Rimantadine, Arbidol, Laninamivir, Peramivir, Vitamin D, and an interferon.

15. The method of any one of claims 12 to 14, wherein the second therapeutic agent is administered before, after, or concurrently with administering the nucleic acid molecule or the L7Ae protein.

16. A method for reducing influenza virus replication in a subject or a biological sample thereof, comprising (i) administering to the subject a nucleic acid molecule comprising a polynucleotide encoding an L7Ae protein or a fragment/variant thereof or administering to the subject the L7Ae protein or a fragment/variant thereof; or (ii) contacting the biological sample with the nucleic acid molecule or the L7Ae protein or fragment/variant thereof.

17. The method of claim 16, wherein the nucleic acid molecule or the L7Ae protein is administered prophylactically or therapeutically.

18. The method of claim 16 or 17, wherein: (i) the L7Ae protein comprises an amino acid sequence having at least 75% identity to any one of SEQ ID NOs: 1-10, or comprises an amino acid sequence of SEQ ID NOs: 1-10; or (ii) the L7Ae protein comprises an amino acid sequence having at least 63% identity to the amino acid sequence of SEQ ID NO: 4 or SEQ ID NO: 1, or comprises the amino acid sequence of SEQ ID NO: 4 or SEQ ID NO: 1, (a) optionally wherein the L7Ae protein comprises an I or L at position 88, an E or N at position 89, or a V at position 90, or combinations thereof, wherein the position is relative to SEQ ID NO:1, (b) optionally wherein the L7Ae protein comprises an I or L at position 88, an E or N at position 89, and a V at position 90, wherein the position is relative to SEQ ID NO:1, (c) optionally wherein the L7Ae protein comprises an I at position 88, an E at position 89, and a V at position 90, (d) optionally wherein the L7Ae protein comprises an I at position 88, an N at position 89, and a V at position 90, (e) optionally wherein the L7Ae protein comprises an L at position 88, an N at position 89, and a V at position 90, (f) optionally wherein the L7Ae protein comprises an L at position 88, an E at position 89, and a V at position 90, (g) optionally wherein the L7Ae protein does not comprise a R at position 90, (h) optionally wherein the L7Ae protein further comprises one or more of amino acid residues N33, E34, K37, R41, and K79, wherein the position is relative to SEQ ID NO:1, or (i) optionally wherein the L7Ae protein comprises an I or L at position 88, an E or N at position 89, a V at position 90, an N at position 33, an E at position 34, a K at position 37, an R at position 41, and a K at position 79, wherein the position is relative to SEQ ID NO:1.

19. The method of any one of claims 16 to 18, wherein the polynucleotide comprises a nucleotide sequence having at least 75% identity to any one of SEQ ID NOs: 11-15, or comprises a nucleotide sequence of SEQ ID NOs: 11-15.

20. The method of any one of claims 16 to 19, wherein the nucleic acid molecule comprises a viral vector.

21. The method of claim 20, wherein the viral vector comprises an adeno-associated viral vector, lentiviral vector or adenoviral vector.

22. The method of claim 21, wherein the adeno-associated viral vector is selected from the group consisting of AAV1, AAV2, AAV3, AAV4, AAV5, AAV6, AAV7, AAV8, AAV9, AAV10, AAV11, AAV 12, AAV13, AAV rh74, and recombinant subtypes thereof.

23. The method of any one of claims 16 to 22, wherein the influenza virus infection or influenza virus disease is associated with an influenza A virus, an influenza B virus, an influenza C virus, or an Isavirus.

24. The method of claim 23, wherein the influenza A virus comprises a serotype selected from the group consisting of H1N1, H2N2, H3N1, H3N2, H5N1, H7N7, H1N2, H9N2, H7N2, H7N3, H10N7, H7N9, and H6N1.

25. The method of any one of claims 16 to 24, wherein the subject is a mammal, a fish, or an avian.

26. The method of any one of claims 16 to 25, wherein the subject is human.

27. The method of any one of claims 16 to 26, comprising administrating to the subject or contacting with the biological sample thereof with a second therapeutic agent or therapy.

28. The method of claim 27, wherein the second therapeutic agent comprises an antiviral agent.

29. The method of claim 28, wherein the antiviral agent is selected from the group consisting of Oseltamivir, Zanamivir, Amantadine, Rimantadine, Arbidol, Laninamivir, Peramivir, Vitamin D, and an interferon.

30. The method of any one of claims 27 to 29, wherein the second therapeutic agent is administered before, after, or concurrently with the nucleic acid molecule or the L7Ae protein.

31. A method for inhibiting splicing of one or more influenza virus mRNA segments in a subject or a biological sample thereof, comprising (i) administering to the subject a nucleic acid molecule comprising a polynucleotide encoding an L7Ae protein or a fragment/variant thereof or administering to the subject the L7Ae protein or fragment/variant thereof; or (ii) contacting the biological sample with the nucleic acid molecule or the L7Ae protein or fragment/variant thereof.

32. The method of claim 31, wherein the one or more influenza virus mRNA segments comprise M2 mRNA segment, NS2 mRNA segment, or both.

33. The method of claim 31 or 32, wherein: (i) the L7Ae protein comprises an amino acid sequence having at least 75% identity to any one of SEQ ID NOs: 1-10, or comprises an amino acid sequence of SEQ ID NOs: 1-10; or (ii) the L7Ae protein comprises an amino acid sequence having at least 63% identity to the amino acid sequence of SEQ ID NO: 4 or SEQ ID NO: 1, or comprises the amino acid sequence of SEQ ID NO: 4 or SEQ ID NO: 1, (a) optionally wherein the L7Ae protein comprises an I or L at position 88, an E or N at position 89, or a V at position 90, or combinations thereof, wherein the position is relative to SEQ ID NO:1, (b) optionally wherein the L7Ae protein comprises an I or L at position 88, an E or N at position 89, and a V at position 90, wherein the position is relative to SEQ ID NO:1, (c) optionally wherein the L7Ae protein comprises an I at position 88, an E at position 89, and a V at position 90, (d) optionally wherein the L7Ae protein comprises an I at position 88, an N at position 89, and a V at position 90, (e) optionally wherein the L7Ae protein comprises an L at position 88, an N at position 89, and a V at position 90, (f) optionally wherein the L7Ae protein comprises an L at position 88, an E at position 89, and a V at position 90, (g) optionally wherein the L7Ae protein does not comprises a R at position 90, (h) optionally wherein the L7Ae protein further comprises one or more of amino acid residues N33, E34, K37, R41, and K79, wherein the position is relative to SEQ ID NO:1, or (i) optionally wherein the L7Ae protein comprises an I or L at position 88, an E or N at position 89, a V at position 90, an N at position 33, an E at position 34, a K at position 37, an R at position 41, and a K at position 79, wherein the position is relative to SEQ ID NO:1.

34. The method of any one of claims 31 to 33, wherein the polynucleotide comprises a nucleotide sequence having at least 75% identity to any one of SEQ ID NOs: 11-15, or comprises a nucleotide sequence of SEQ ID NOs: 11-15.

35. The method of any one of claims 31 to 34, wherein the nucleic acid molecule comprises a viral vector.

36. The method of claim 35, wherein the viral vector comprises an adeno-associated viral vector, lentiviral vector or adenoviral vector.

37. The method of claim 36, wherein the adeno-associated viral vector is selected from the group consisting of AAV1, AAV2, AAV3, AAV4, AAV5, AAV6, AAV7, AAV8, AAV9, AAV10, AAV11, AAV 12, AAV13, AAV rh74, or recombinant subtypes thereof.

38. The method of any one of claims 31 to 37, wherein the influenza virus infection or influenza virus disease is associated with an influenza A virus, an influenza B virus, an influenza C virus, or an Isavirus.

39. The method of claim 38, wherein the influenza A virus comprises a serotype selected from the group consisting of H1N1, H2N2, H3N1, H3N2, H5N1, H7N7, H1N2, H9N2, H7N2, H7N3, H10N7, H7N9, and H6N1.

40. A vector comprising a polynucleotide encoding a L7Ae protein or a fragment/variant thereof.

41. The vector of claim 40, wherein: (i) the L7Ae protein comprises an amino acid sequence having at least 75% identity to any one of SEQ ID NOs: 1-10, or comprises an amino acid sequence of SEQ ID NOs: 1-10; or (ii) the L7Ae protein comprises an amino acid sequence having at least 63% identity to the amino acid sequence of SEQ ID NO: 4 or SEQ ID NO: 1, or comprises the amino acid sequence of SEQ ID NO: 4 or SEQ ID NO: 1, (a) optionally wherein the L7Ae protein comprises an I or L at position 88, an E or N at position 89, or a V at position 90, or combinations thereof, wherein the position is relative to SEQ ID NO:1, (b) optionally wherein the L7Ae protein comprises an I or L at position 88, an E or N at position 89, and a V at position 90, wherein the position is relative to SEQ ID NO:1, (c) optionally wherein the L7Ae protein comprises an I at position 88, an E at position 89, and a V at position 90, (d) optionally wherein the L7Ae protein comprises an I at position 88, an N at position 89, and a V at position 90, (e) optionally wherein the L7Ae protein comprises an L at position 88, an N at position 89, and a V at position 90, (f) optionally wherein the L7Ae protein comprises an L at position 88, an E at position 89, and a V at position 90, (g) optionally wherein the L7Ae protein does not comprise a R at position 90, (h) optionally wherein the L7Ae protein further comprises one or more of amino acid residues N33, E34, K37, R41, and K79, wherein the position is relative to SEQ ID NO:1, or (i) optionally wherein the L7Ae protein comprises an I or L at position 88, an E or N at position 89, a V at position 90, an N at position 33, an E at position 34, a K at position 37, an R at position 41, and a K at position 79, wherein the position is relative to SEQ ID NO:1.

42. The vector of claim 40 or 41, wherein the polynucleotide comprises a nucleotide sequence having at least 75% identity to any one of SEQ ID NOs: 11-15, or comprises a nucleotide sequence of SEQ ID NOs: 11-15.

43. The vector of any one of claims 40 to 42, wherein the vector is a viral vector.

44. The vector of any one of claims 40 to 43, wherein the viral vector is an adeno-associated viral vector, lentiviral vector or adenoviral vector.

45. The vector of any one of claims 40 to 44, wherein the adeno-associated viral vector is selected from the group consisting of AAV1, AAV2, AAV3, AAV4, AAV5, AAV6, AAV7, AAV8, AAV9, AAV10, AAV11, AAV 12, AAV13, AAV rh74, and recombinant subtypes thereof.

46. A host cell comprising the vector of any one of claims 40 to 45.

47. A composition comprising the vector of any one of claims 40 to 45, or the host cell of claim 46.
